(19) United States
(12) Patent Application Publication
(10) Pub. No.: US 2004/0088189 A1
(43) Pub. Date: May 6, 2004

(54) System and method for monitoring , reporting, managing and administering the treatment of a blood component

(76) Inventors: Edmond A. Veome, La Grange, IL (US); Christophe Vermeiren, Jette (BE); Chris Noel Fredericks, Libertyville, FL (US); Samira E. Johnson, Hawthorn Woods, IL (US); Lori Rabe, Oakwood Hills, IL (US); Savvas Merkouriou, Ellington, CT (US); Karen Berthiaume, Nashua, NH (US)

Correspondence Address: James P. Muraff, Wallenstein & Wagner, Ltd., 311 S. Wacker Drive, 53rd Floor, Chicago, IL, 60606-6630, US

(21) Appl. No.: 10/290,035

(22) Filed: Nov. 6, 2002

(51) Int. Cl. A01N 1/02; G06F 17/60; C07K 1/00; C07K 14/00; C07K 16/00; C07K 17/00; A61K 35/14; A61K 35/16

(52) U.S. Cl. 705/2; 424/530; 435/2; 530/382

(57) ABSTRACT

A system and method for treating a biological fluid unit wherein the system ensures proper execution of the treatment by coordinating cooperation among a plurality of components operably connected to the system. The system components include a processor communicating with a memory, a unit identifier, at least one blood component treatment process, and an illumination instrument. The system includes a database stored in and accessible from the memory. The database retains information associated with a blood component, an illumination instrument, and treatment of the blood component utilizing the illumination instrument. A user interface operably connected to the processor includes a graphical screen and a data entry device for communicating information related to the blood component treatment to and from the operator.

### RELATED APPLICATIONS

This application claims priority to U.S. Provisional Patent Application Serial No. 60/287,122, entitled Automated Blood Tracking System and Interface, filed 28 Apr. 2001, and U.S. patent application Ser. No. 09/864,926, entitled System and Method for Automating the Workflow in a Blood Collection Facility, filed 24 May 2001. These applications are incorporated herein by reference.

### DESCRIPTION

1. Technical Field

The present invention generally relates to processing and treating biological fluids, such as blood and blood components. More specifically, the present invention is directed to monitoring, reporting, managing and/or administering a pathogen inactivation process applied to biological fluids.

2. Background of the Invention

Pathogen inactivation, PI, is a treatment applied to biological fluids, e.g., blood and blood components as part of a production process prior to distribution to a healthcare system for patient transfusion. Various types of pathogens (viral, bacterial, and parasitic) are inactivated by the interruption of DNA/RNA replication. DNA containing cells present in the blood component, e.g. leukocytes, may also be inactivated. One example of PI is achieved by introducing a light-activated photochemical, e.g., amotosalen (S-59), into the biological fluid for the purpose of inactivating pathogens that may be present. The biological fluid is then illuminated to activate the photochemical, effectively preventing DNA/RNA replication. After the required dosage of illumination is reached, the remaining photochemical is removed via an adsorption wafer and the biological fluid is ready to be transferred to a final storage container and appropriately labeled for product distribution.

There are a number of facilities/organizations that are equipped to collect blood and blood components, e.g., plasma, platelets, and red cells. Tracking donors, component handling, donor registrations, instrument operability, and the like are important aspects of the blood component collection industry. One such system, U.S. Patent Application Publication No. 2001/0034614 A1, discloses an information management system for an extracorporeal blood collection procedure wherein the system is operably connected to a donor and information is communicated throughout the system for monitoring the blood collection process.

The biological fluid treatment industry has long recognized a void in automated management of information related to the treatment of blood and blood components irrespective of a donor's intimate involvement with the system, e.g., extracorporeal blood collection process. Additionally, PI (pathogen inactivation) methods do not practically allow for automated quality control. The data resulting from the treatment process is not centrally stored, easily accessible, or automatically tracked. Thus, process documentation as a quality assurance mechanism can be burdensome and error-prone.

While the prior art apparatuses, systems and methods have generally operated satisfactorily, a need remains for an apparatus and system that provides an automated, reliable process control or process audit mechanism capable of managing a pathogen inactivation treatment of a biological fluid. It is also desirable for such a system to be capable of interacting with various instruments utilized in the treatment and generating overall status reports for reviewing and monitoring the treatment of blood components. Further, it is also desirable that the system be electronic-based rather than the typical paper-based record-keeping utilized in the industry.

The present invention is provided to solve these and other problems.

### SUMMARY OF THE INVENTION

The present invention is a method, system, and program for monitoring, reporting, managing and/or administering the treatment of a blood component. As used herein, blood product treatment refers to treatment after collection has taken place, such as treatment after apheresis processing has been completed. The system includes a processor 8 and a memory 130 operably connected to the processor. The memory 130 can have software therein, which can include a module for monitoring, a module for reporting, a module for managing, and a module for administering a biological fluid treatment. A treatment instrument communicates with the processor and memory 130 for performing these functions. An interface is communicably connected to the processor, and includes a means for entering information and a means for displaying information. The information is related to the biological fluid treatment wherein the memory 130 and the interface cooperate to manage the biological fluid treatment.

In one embodiment, the present invention is a method for managing and administering a blood component treatment. A blood component treatment requires execution of a plurality of complicated process steps. For example, in one type of a blood component treatment, the pathogen inactivation process can include the following steps: pooling individual units into a single larger blood component unit; registration of a new blood component unit; pre-verification; sterile docking; post-verification of a match between the product and the pathogen inactivation kit or processing set; illumination; agitation; and evaluation.

The present invention automatically documents and captures data at each processing step, and helps ensure that each of the steps have been properly executed and administered. Information generated throughout the PI process is automatically captured, stored, and archived. Further, the present invention allows for easy accessibility to data for indicating the treatment status of a biological fluid product, e.g., treatment problems, identification of successfully completed treatment steps, other treatment results, inventory, operator interaction history, equipment maintenance logs, etc. Further, the present invention is a system that provides a mechanism for automatically transferring blood component treatment information directly to a host site, thus reducing data errors. The present invention can also extract blood component treatment data from the host system site. Likewise, the present invention can further send blood component treatment data to the host system.

In an alternate embodiment, the present invention is a computer-readable medium utilized in a system comprising an application to facilitate the monitoring, reporting, managing, and/or administration of a biological fluid treatment. The medium optionally includes one or more segments, including a first segment for verifying compatibility of a biological fluid unit with a pathogen inactivation kit or processing set including fluid volume and composition. A second segment receives information from a pathogen inactivation instrument utilized during treatment and verifies that the elapsed time between the biological fluid collection and the treatment remains within established or predetermined time parameters. This segment also captures information relating to an amount of illumination exposure substantially transmitted from the pathogen inactivation instrument to the biological fluid. A third segment enables communication between a memory 130 for storing treatment status and history and a system interface.

Other features and advantages of the invention will be apparent from the following specification taken in conjunction with the following drawings.

### BRIEF DESCRIPTION OF THE DRAWINGS

FIG. 1 is a block diagram of one embodiment of a pathogen inactivation method.

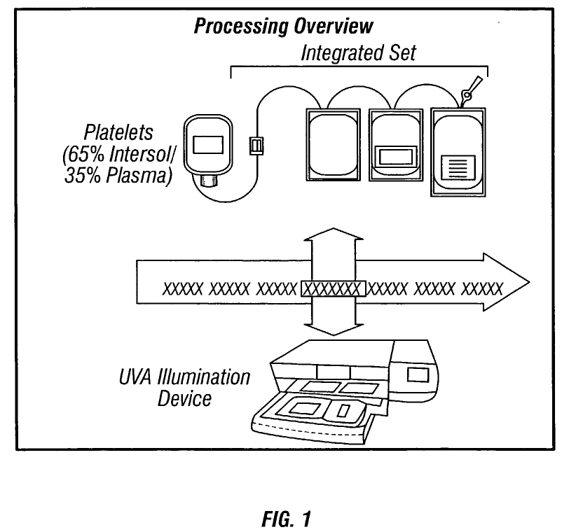

FIG. 2 is a block diagram of one embodiment of a hardware and a software configuration utilized within the present invention.

FIG. 3 is a block diagram of another embodiment of the present invention.

FIG. 4 is a block diagram of a further embodiment of the present invention.

FIG. 5 is a block diagram of one embodiment of a network architecture utilized with the present invention.

FIG. 6 is a flowchart showing the overall process of one embodiment of the present invention.

FIG. 7 is a flowchart showing one embodiment of a unit search process of the present invention.

FIG. 8 is a flowchart showing one embodiment of a registration process of the present invention.

FIG. 9 is a flowchart showing one embodiment of a pre-verification process of the present invention.

FIG. 10 is a flowchart showing one embodiment of a post-verification process of the present invention.

FIG. 11 is a flowchart showing one embodiment of an illumination process of the present invention.

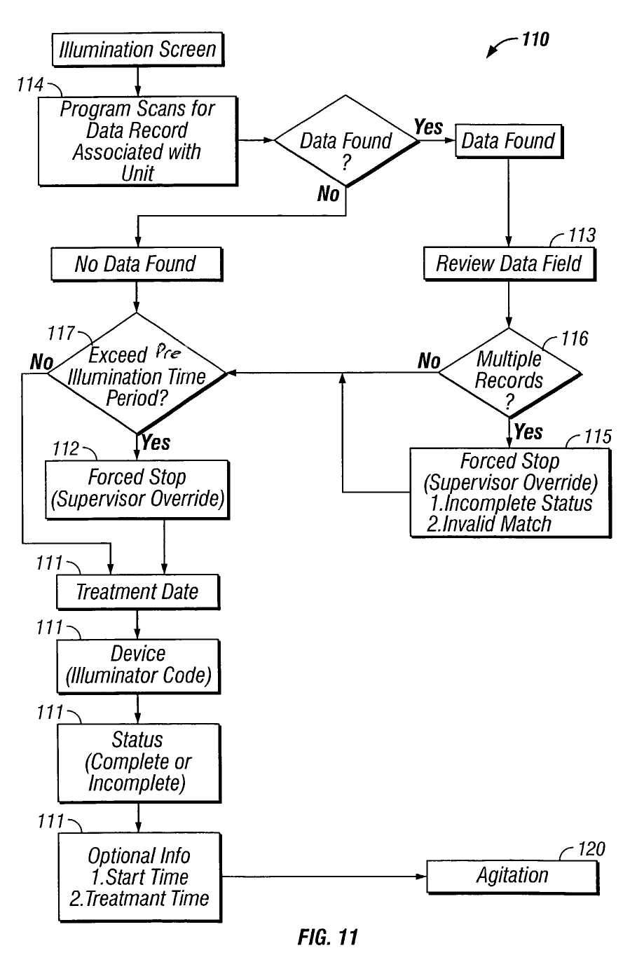

FIG. 12 is a flowchart showing one embodiment of an agitation process of the present invention.

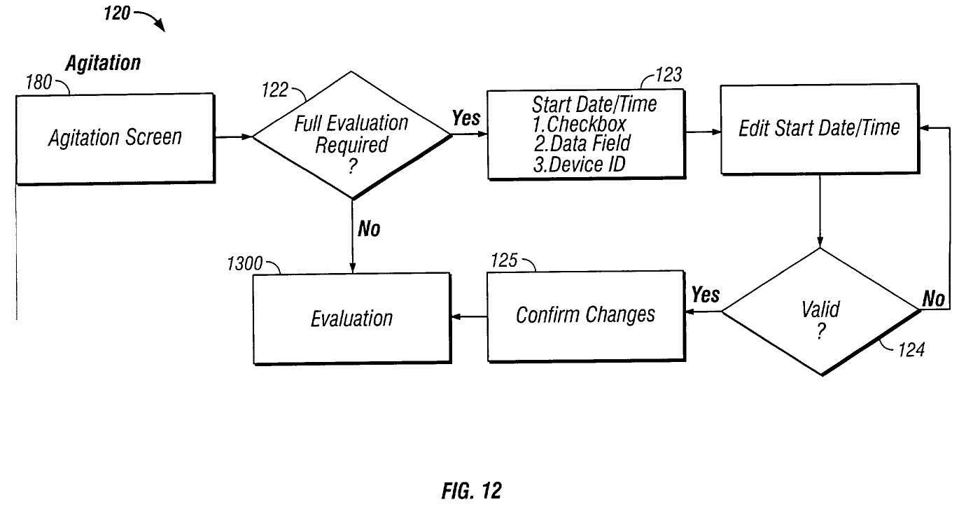

FIG. 13 is a flowchart showing one embodiment of an evaluation process of the present invention.

FIG. 14 is a block diagram of one embodiment of a toolbar functionality of the present invention.

FIGS. 15a and 15b are a flowchart showing one embodiment of a pooling process of the present invention.

FIG. 16 is a flowchart showing one embodiment of a manual stop process of the present invention.

FIG. 17 is a flowchart showing one embodiment of a supervisor override process of the present invention.

FIG. 18a depicts one embodiment of a login screen of the user interface of the present invention.

FIG. 18b depicts one embodiment of a main screen of the user interface of the present invention.

FIG. 18c depicts another embodiment of a main screen of the user interface of the present invention.

FIG. 18d depicts one embodiment of a preprocessing screen of the user interface of the present invention.

FIG. 18e depicts another embodiment of a preprocessing screen of the user interface of the present invention.

FIG. 18f depicts one embodiment of a registration screen of the user interface of the present invention.

FIG. 18g depicts one embodiment of a pre-verification screen of the user interface of the present invention.

FIG. 18h depicts one embodiment of a post-verification screen of the user interface of the present invention.

FIG. 18i depicts another embodiment of a post-verification screen of the user interface of the present invention.

FIG. 18j depicts one embodiment of an illumination screen of the user interface of the present invention.

FIG. 18k depicts another embodiment of an illumination screen of the user interface of the present invention.

FIG. 18l depicts one embodiment of an agitation screen of the user interface of the present invention.

FIG. 18m depicts one embodiment of an evaluation screen of the user interface of the present invention.

FIG. 18n depicts one embodiment of treatment completion status screen of the user interface of the present invention.

FIG. 18o depicts one embodiment of a pooling screen of the user interface of the present invention.

FIG. 18p depicts one embodiment of an agitation list screen of the user interface of the present invention.

FIG. 18q depicts one embodiment of a completion list screen of the user interface of the present invention.

FIG. 18r depicts one embodiment of a reports screen of the user interface of the present invention.

FIG. 18s depicts one embodiment of a history screen of the user interface of the present invention.

FIG. 18t depicts one embodiment of a manual stop screen of the user interface of the present invention.

FIG. 18u depicts one embodiment of a supervisor override screen of the user interface of the present invention.

FIG. 18v depicts one embodiment of a history reports screen of the user interface of the present invention.

FIG. 19 is a block diagram depicting one embodiment of a database utilized in the present invention.

### DETAILED DESCRIPTION

While this invention is susceptible to embodiments in many different forms, there are shown in the drawings and will herein be described in detail, preferred embodiments of the invention with the understanding that the present disclosures are to be considered as exemplifications of the principles of the invention and are not intended to limit the broad aspects of the invention to the embodiments illustrated.

The present invention is generally directed to an apparatus, system, method, and computer readable medium for monitoring, reporting, managing and/or administering treatment of a biological fluid. The system includes various treatment devices which are networked with a central server. The treatment devices can include, but are not limited to, existing biological fluid treatment instruments, such as the pathogen inactivation instruments described in U.S. patent application Ser. Nos. 09/325,325 and 09/325,599, which are assigned to Baxter International, Inc. The teachings from the applications are incorporated herein by reference. Execution of the method is facilitated via retrieval and processing of one or more of the operator data, soft good and corresponding blood component and associated data, and instrument data.

An instrument for treating a biological fluid is referred to herein generally as an illuminator or light box. Please refer to the referenced U.S. patent applications (Ser. Nos. 09/325,325 and 09/325,599) for a detailed description. The illuminator may be used for treating different materials for a variety of purposes. The illuminator is particularly useful in the treatment of biological fluids. As used herein, biological fluid refers to any fluid that is found in, or that may be introduced into the body including, but not limited to, blood, blood components, and blood products. Further, a “blood product” refers to whole blood or a component of whole blood such as red blood cells, white blood cells, platelets, plasma, or any combination of one or more of such components that have been separated from whole blood.

One specific use of the illuminator is for the pathogen inactivation (PI) treatment of a biological fluid unit wherein the unit is combined with a photochemical agent for activation when subjected to light. Such photochemical or photoactive agents are used in the inactivation of viruses, bacteria, parasites and other contaminants C collectively referred to herein as “pathogens.” During a pathogen inactivation treatment, a light-activated agent inactivates pathogens that may be present in the blood product. Typically, the collected blood product in its container is called a unit. These units are then treated using a PI process.

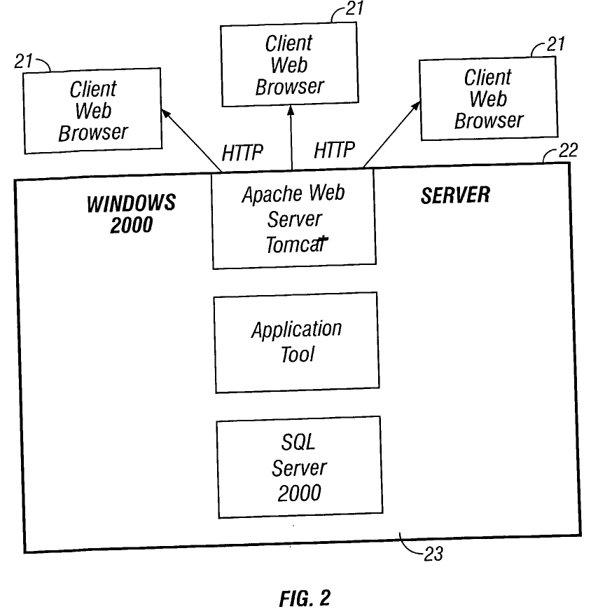

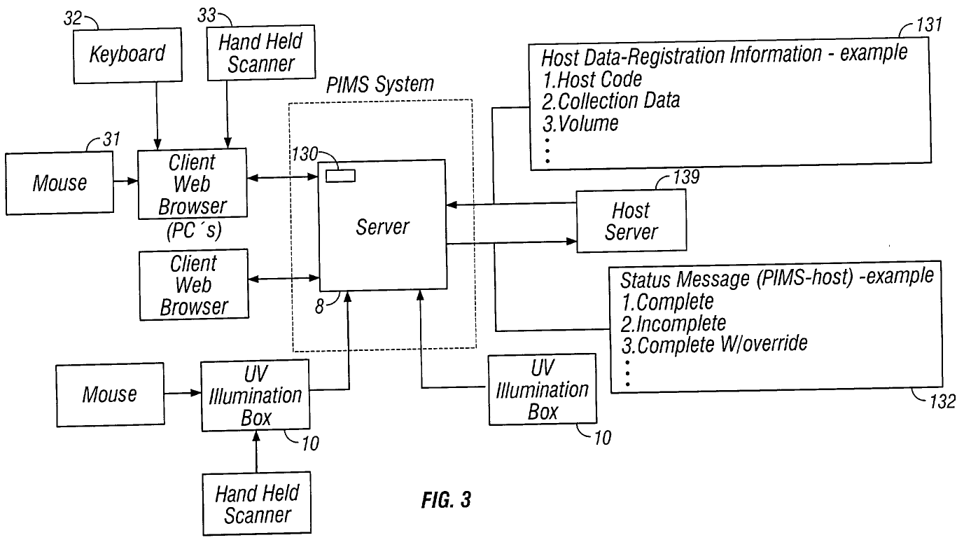

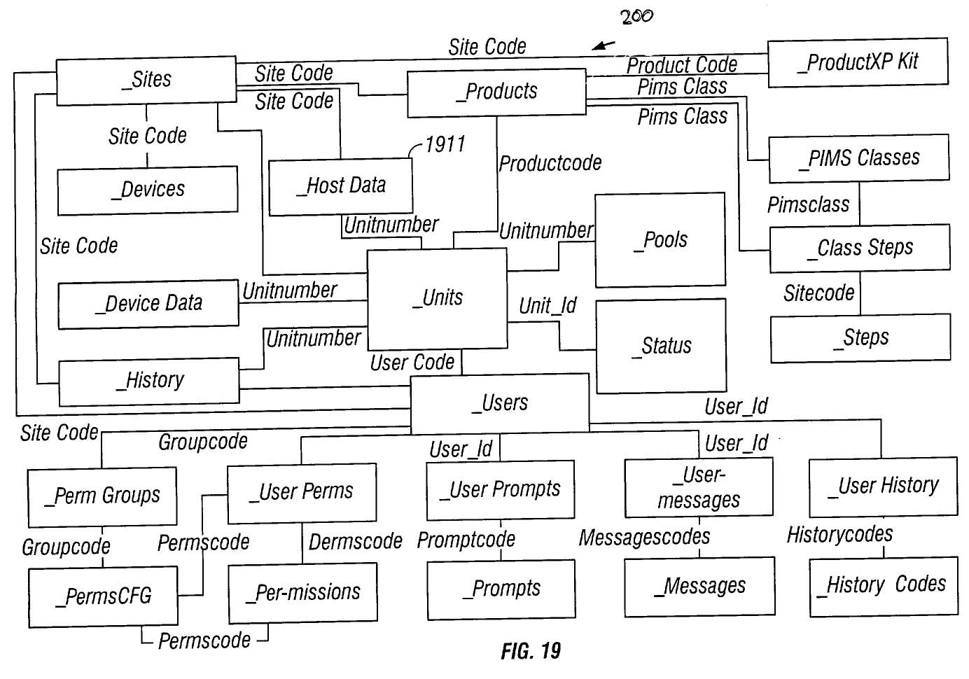

As seen in FIGS. 2, 3, and 4, one embodiment of the present invention utilizes a user interface 126 running on a web browser 21. The interface 126 is operably connected to a processor 8, a memory 130, and a plurality of instruments 10. The user interface 126 is an application component that facilitates communication with operator personnel running the treatment process. The memory 130 can be, but is not limited to, the following types: cache, ROM, RAM, high-speed memory, flash memory, hard disk, floppy disk, and network attached storage. The memory 130 can be used to store information in a database 23 having various tables. FIG. 19 depicts one embodiment of a database that can be utilized in the present invention. FIGS. 20a-20d are examples of various portions of the database shown in FIG. 19. The tables are linked to each other utilizing a particular data schema 200 and are accessible using a set of database communication components 140 and web browser 21.

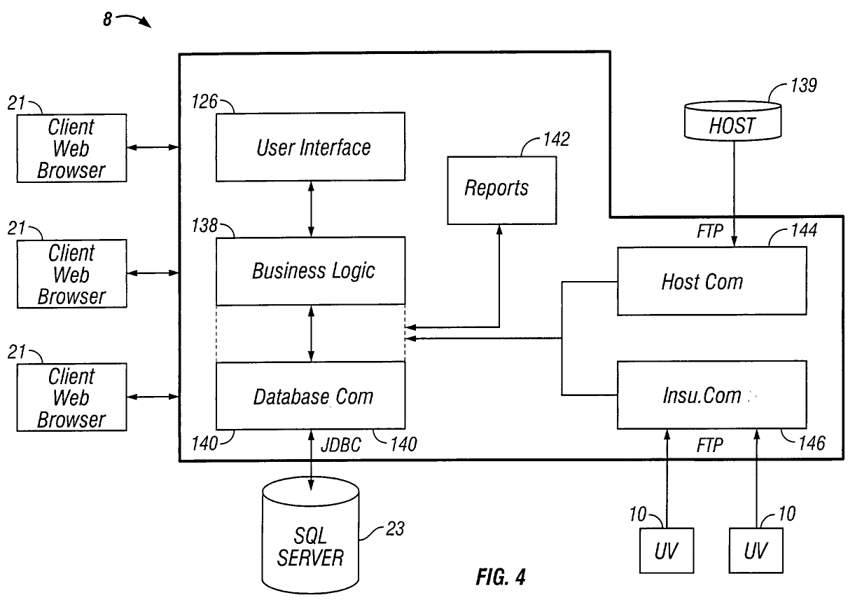

FIG. 4 shows that one embodiment of the present invention further includes a plurality of modules: Business Logic 138, Database Communications 140, Reports 142, Host Communications 144, and Instrument Communications 146.

The business logic module 138 evaluates the user, host, and instrument entries. In one embodiment of the present invention, the business logic 138 components and database 140 components are developed in Java and communicate with Microsoft SQL's server 23 through JDBC (Java Data Base Connectivity protocol) components 147.

The database communications module 140 provides for communication with the SQL server database 23 to store and retrieve required data. The database communication component 140 also holds the SQL strings that will be sent to the SQL server 23 to perform storage and retrieval functions.

The reports module can generate reports from each particular step in the pathogen inactivation treatment. For example, a completion list report provides a list of illuminators that have completed the blood component treatment process. Process reports 142 related to the pathogen inactivation procedure can also be created.

The host communications module 144 provides protocols for communication with a database in a Host server 139 retrieving data fields 131 such as host code, collection data, and unit volume. In one embodiment, the module reads the files sent by the Host via a File Transfer Protocol (FTP) and stores the contents to a database host data table. Other types of transfer protocols can be used as well. After completion of the blood treatment process, a status message 132 is sent back to the Host server 139. In one embodiment of the present invention, the status message 132 could be of type complete, incomplete, or override.

In one embodiment, the instrument communications module 146 reads the files sent by the illumination instrument 10 using FTP and stores the contents in the device data table. The Host and instrument components poll the directory that the Microsoft Windows FTP server stores files, and opens and parses the data sent by the external Host 139 and instrument devices 10. The data is then stored in the database 23. All data searches, updates, and insertions are performed using Transact Standard Query Language (TSQL) strings implemented by the application. Other types of database queries can be used as well. The Standard Query Language (SQL) server connection strings will also be implemented by the application. The instrument communications module interacts with the illumination instrument 10 wherein the length of time for which a quantity of joules applied by the device can be monitored. Preferably, the system is capable of, but not limited to, being configured to operate in the range of 5-10 simultaneous users and 2-4 instruments to 100 simultaneous users and 20 instruments.

The server/client hardware, operating system (OS), and development platform cooperate to allow scaling of the system to support both small and large implementations. Preferably, the processor comprises at least two 1 GHz processors, scaleable to eight, that enable the system to operate properly. Memory 130 is scaleable between at least 1 and 4 GB. The drivers are redundantly configured wherein failure of one driver is adequately supported by another. Ethernet is utilized as the network protocol facilitating communication with the Host's and the network's devices. Other types of networks and arrangements can be used, such as WANs, LANs, Internet, and UPN. Various media accessing devices are operably connected to the system, e.g., tape drive, CD-ROM, floppy disk; along with accompanying display and data entry devices, e.g., keyboard 32, mouse 31, modem, bar-code reader 33, etc.

In one embodiment, the processor OS provides multiple user capability and various language installation options. Thin or fat clients can be utilized with the present invention to support connection via TCP/IP, and HTTP network protocols. The invention contemplates other network configurations, including WAN, LAN, Internet, and VPN.

As shown in FIG. 2, one embodiment of the present invention utilizes a development architecture based on a browser-based thin-client architecture 21 and a 3-tier Distributed Internet Application (DNA-based) server application architecture 22. The clienUserver system follows the standards of Object Oriented Programming (OOP). The server application components present the user interface (UI) 126 to the client machines 15 through Hyper Text Markup Language (HTML) pages configured using Java Server Pages (JSP-based) Java code. The system resides on a server and communicates with the user via the web browser 21 on a client computer. Components are developed in Java and communicate with the server via Java Data Base Connectivity (JDBC). Client machines 15 can include, but are not limited to, desktops, laptops, personal digital assistants, cellular phones, wireless pagers, digital tablets, and other servers.

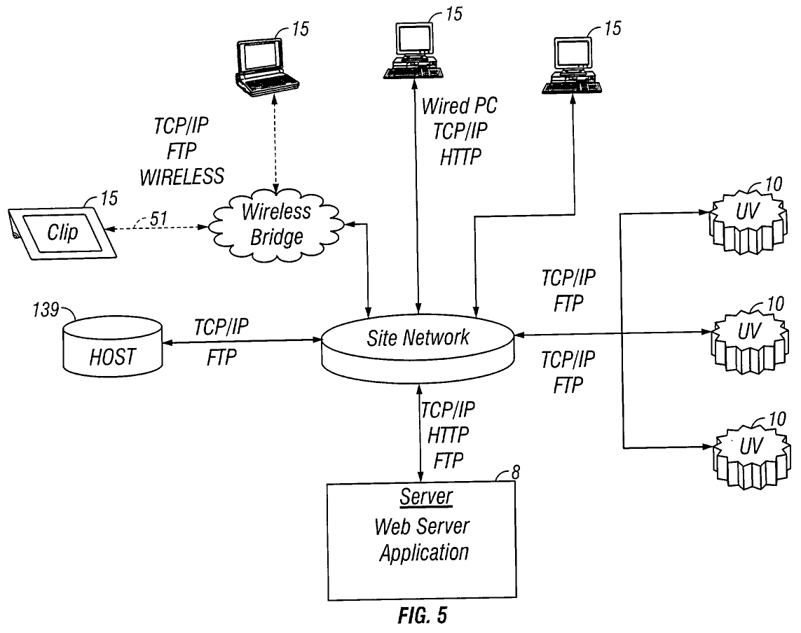

Referring to FIG. 5, preferably, in one embodiment the system's network architecture 16 is based on TCP/IP utilizing Socket, FTP, and HTTP protocols 51. HTML is used to display the interfaces on a client machine 's browser 21 using Java Server Pages (JSP) processes implemented from a standard unix-based server 24. It is further contemplated by the present invention that multiple languages will be supported through strings contained in language-specific fields in the SQL server one field is assigned to each language. Most alpha strings are stored using Unicode compatible fields. A Unicode compatible field is one that is compatible with the Unicode Worldwide Character Standard, which is a system that allows the interchange, processing, and display of the written text of diverse languages, including classical and historical texts. Other types of fields can be used as well. Multiple language support also requires multilingual support by both the server and client operating system and hardware components.

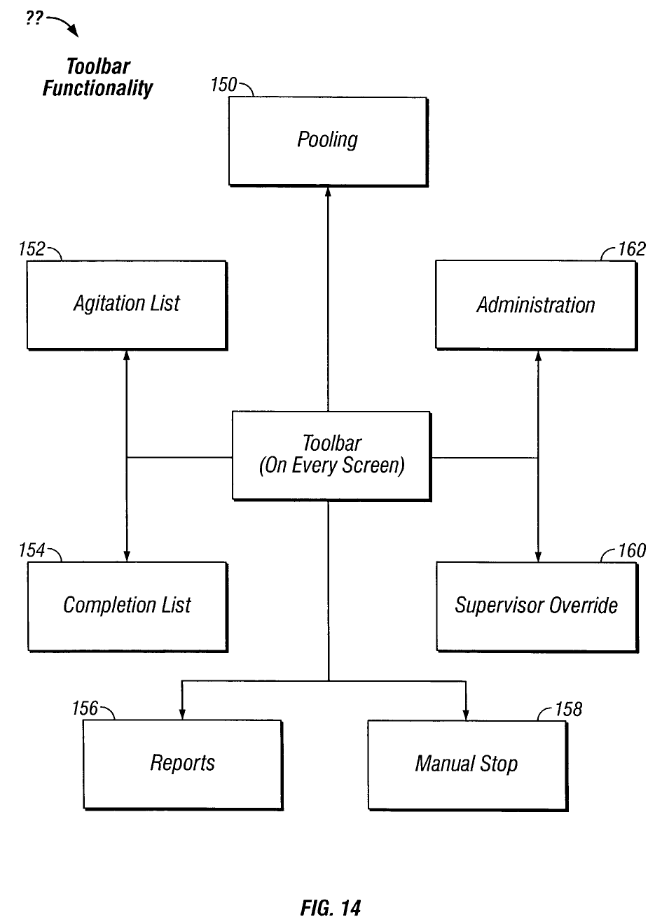

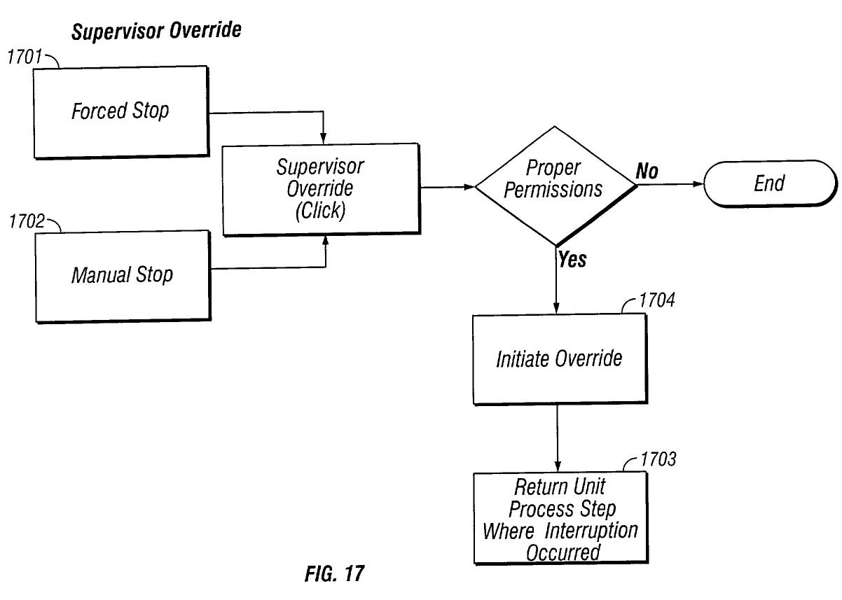

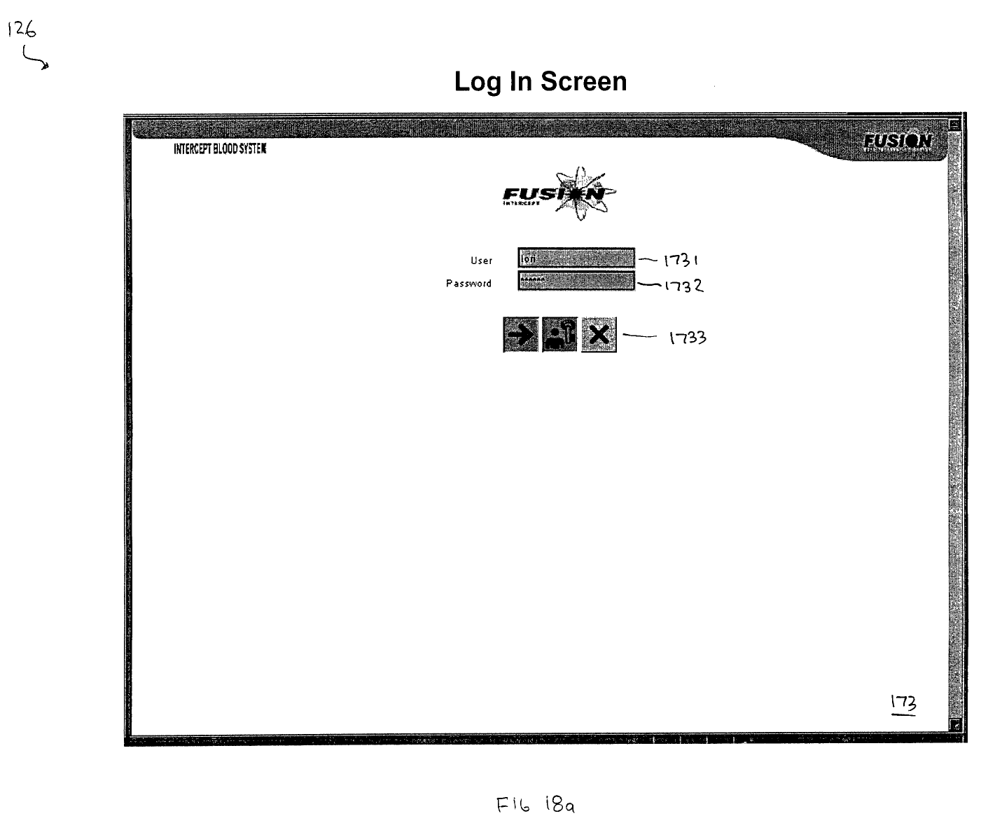

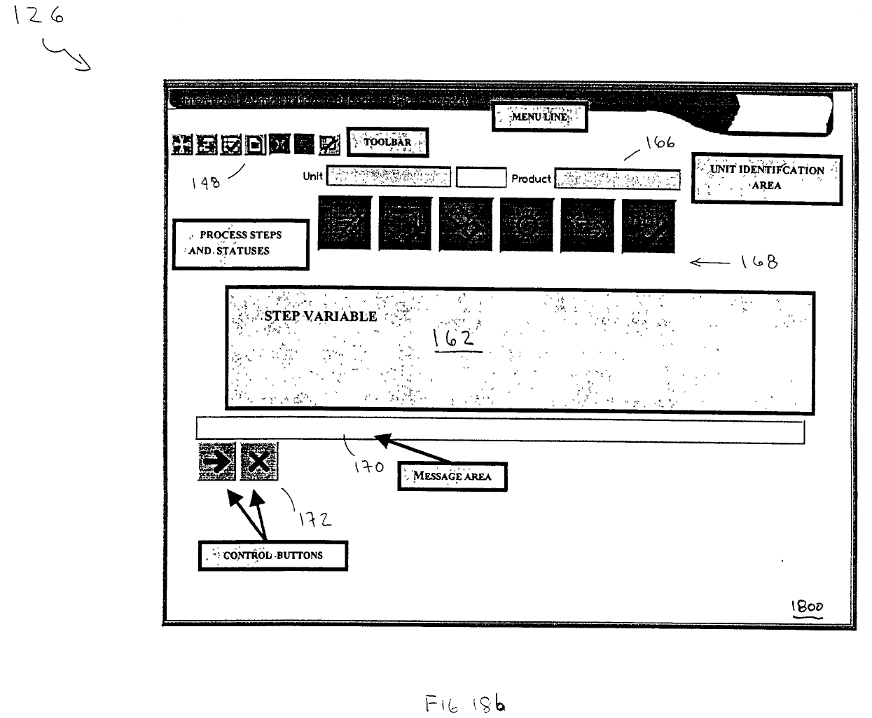

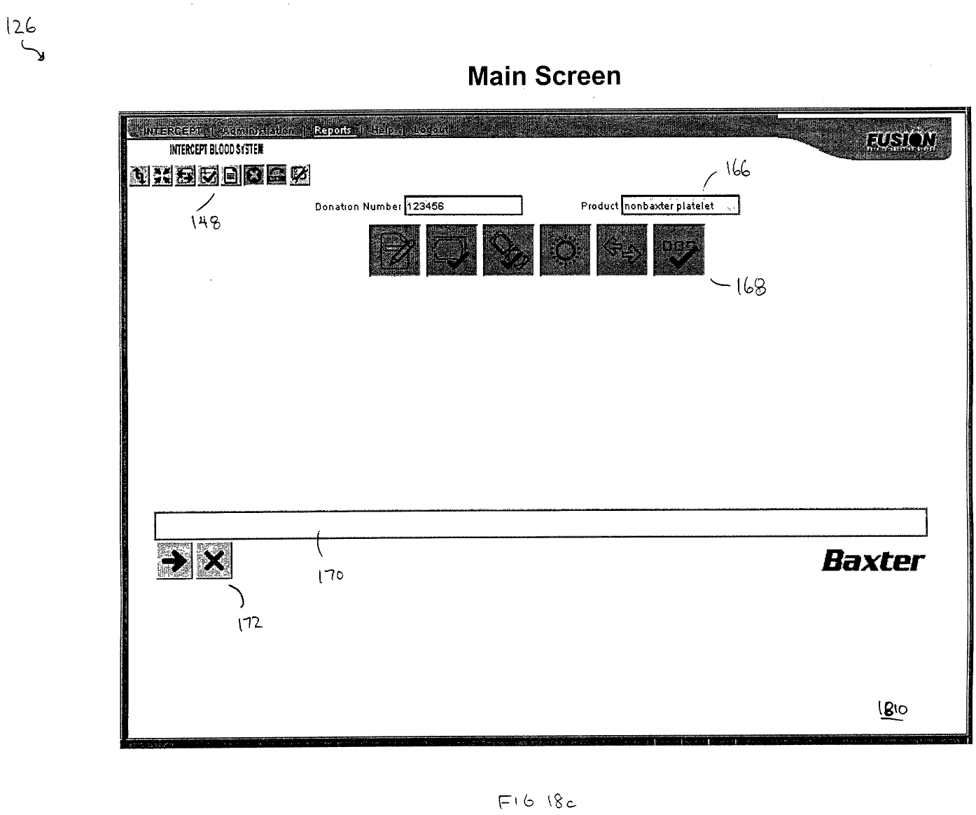

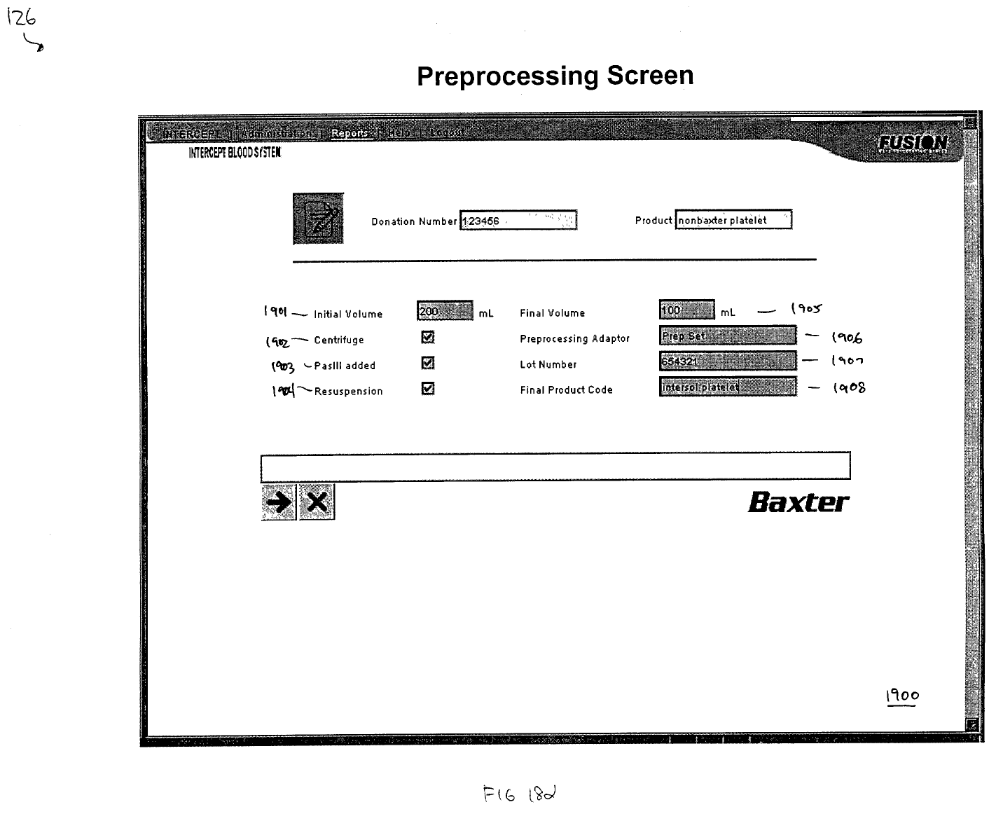

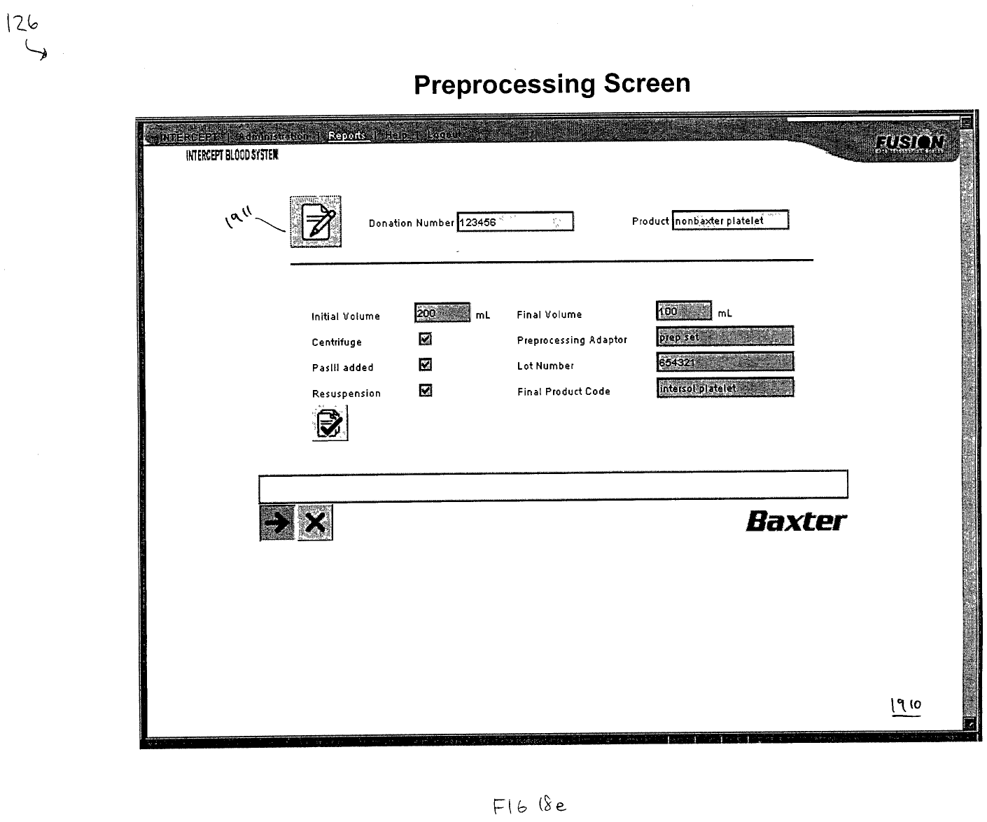

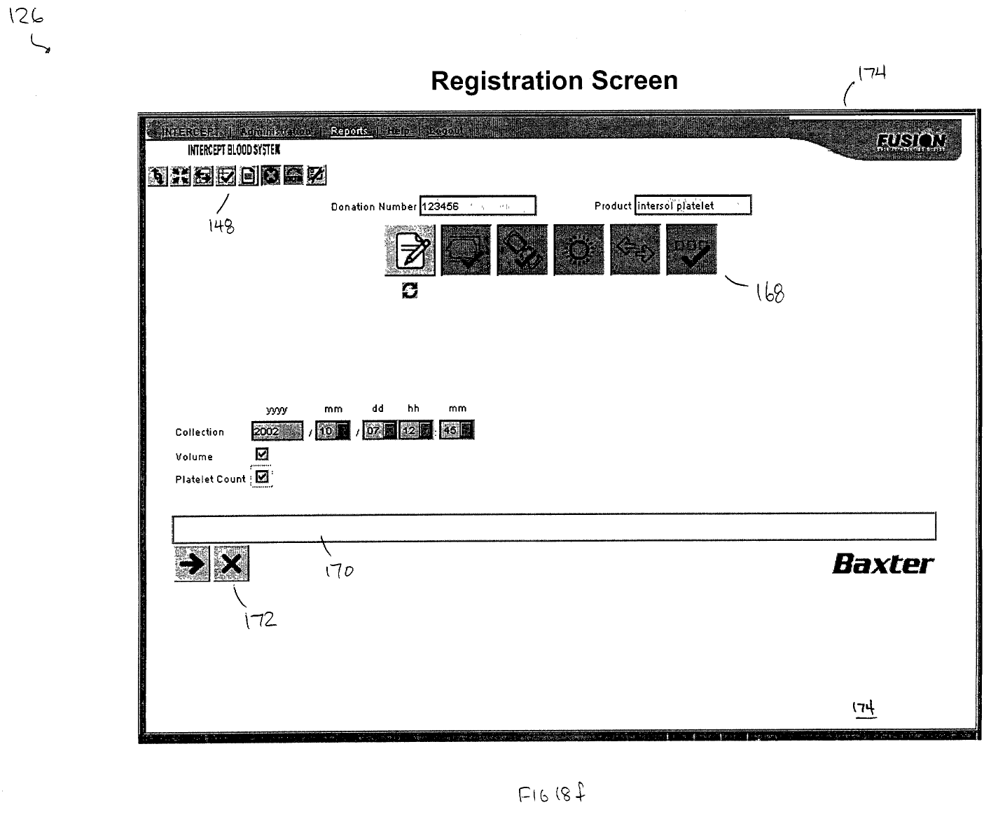

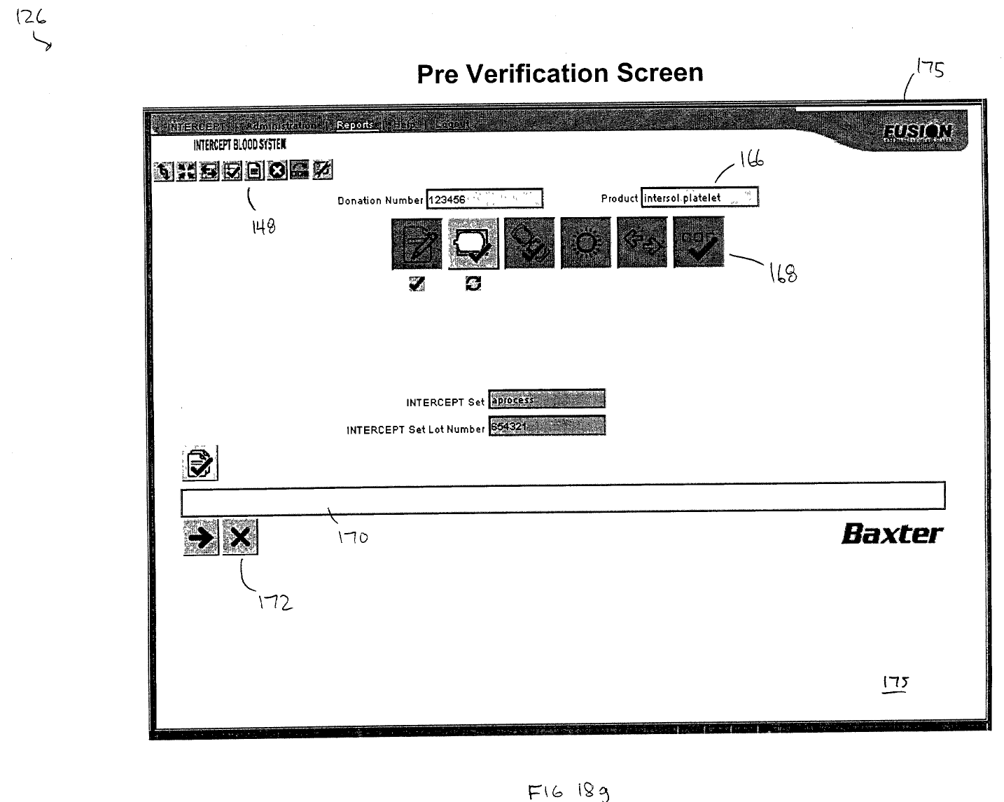

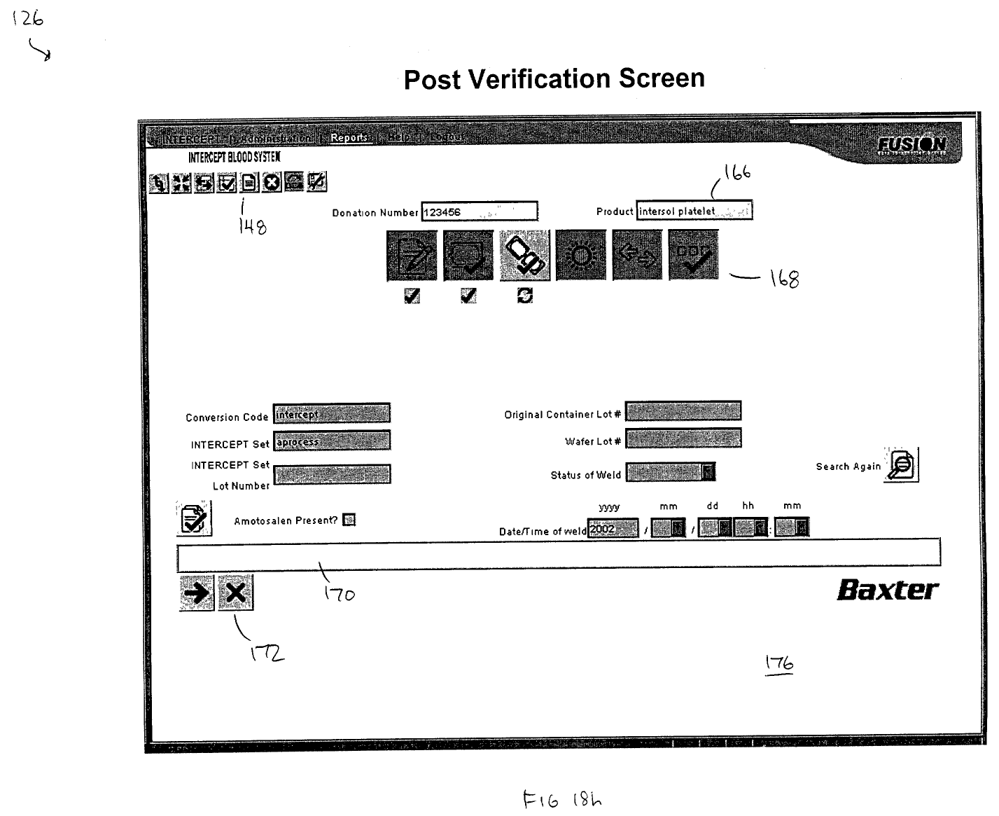

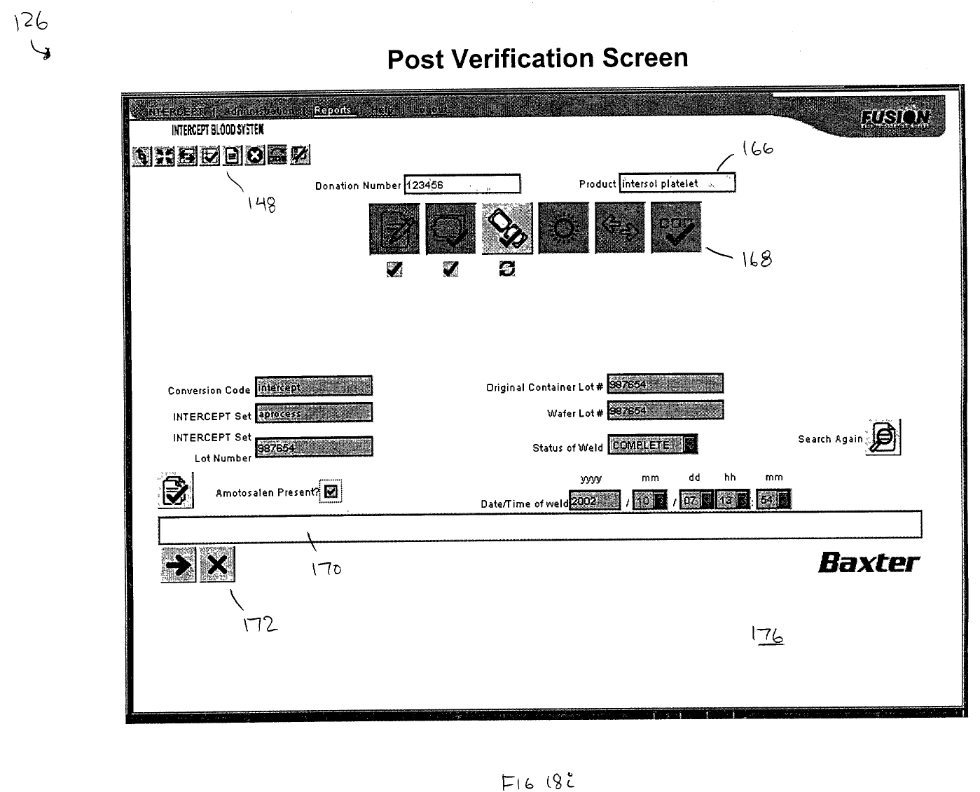

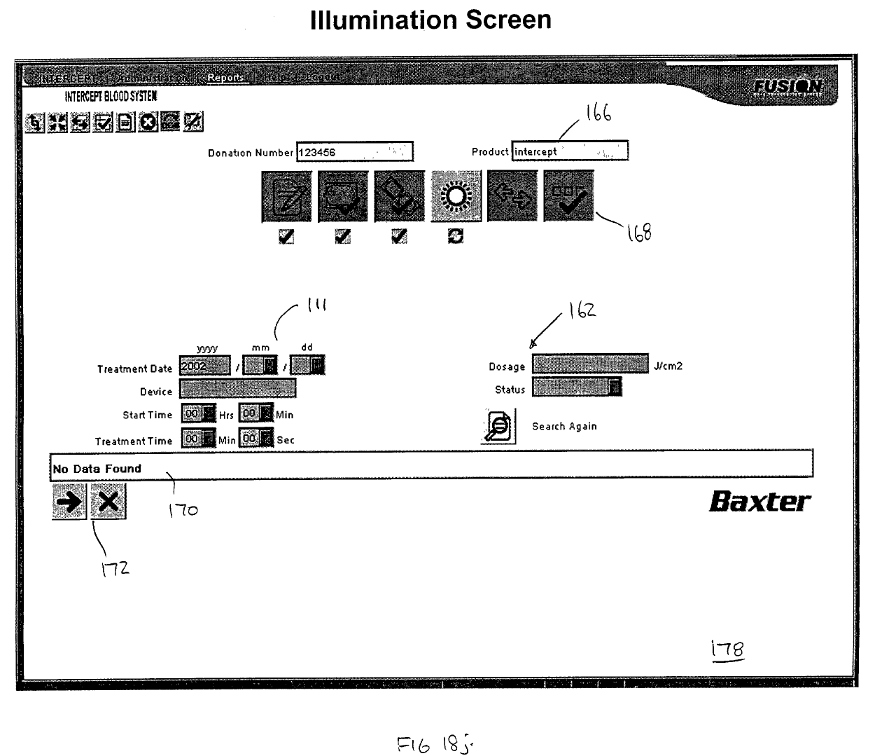

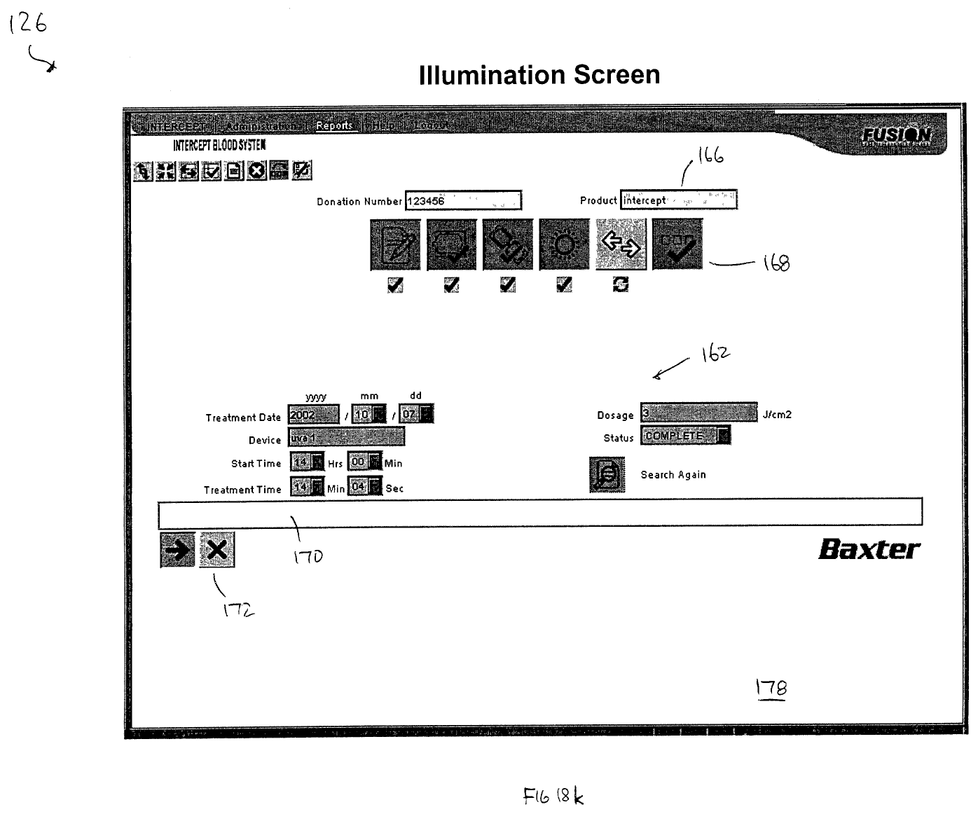

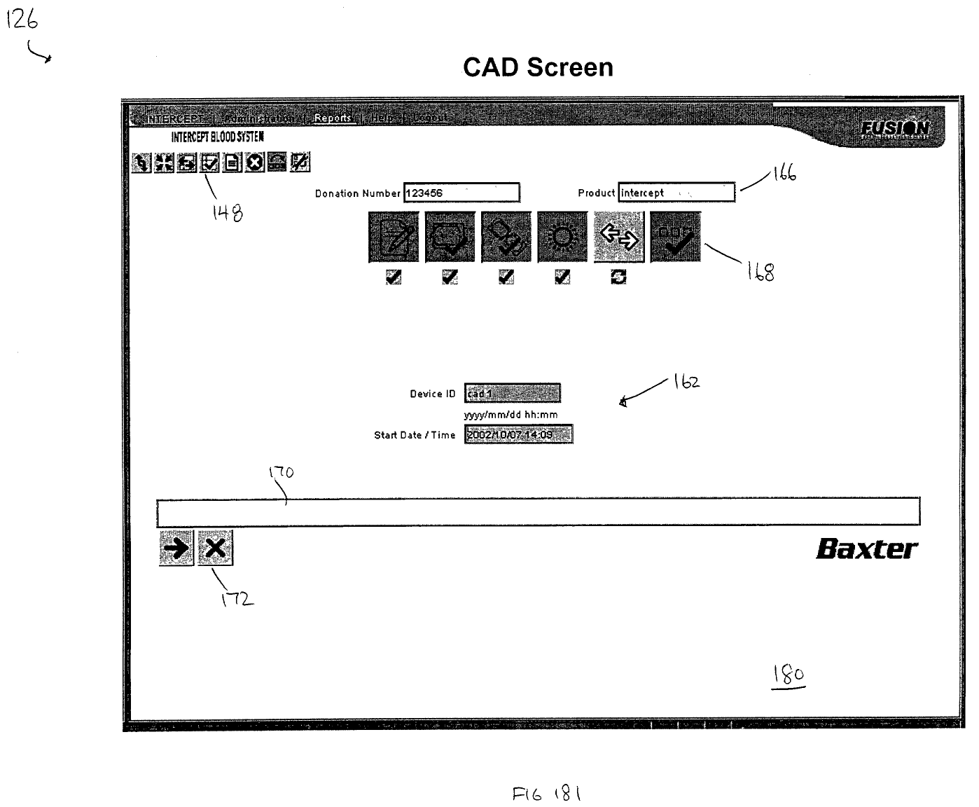

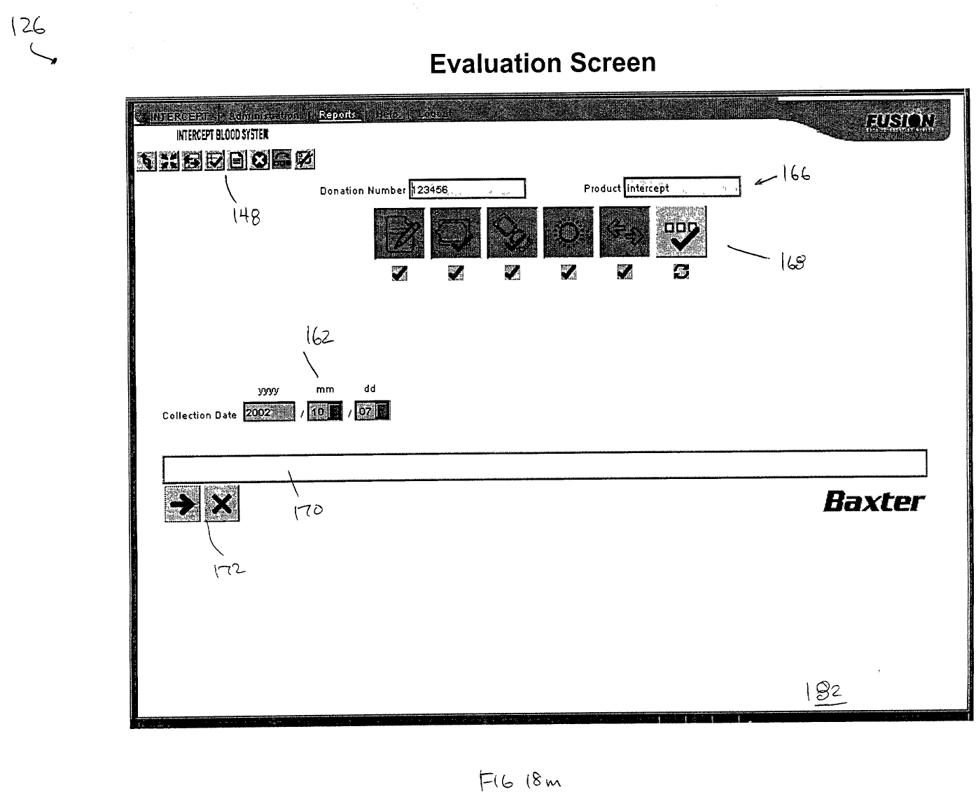

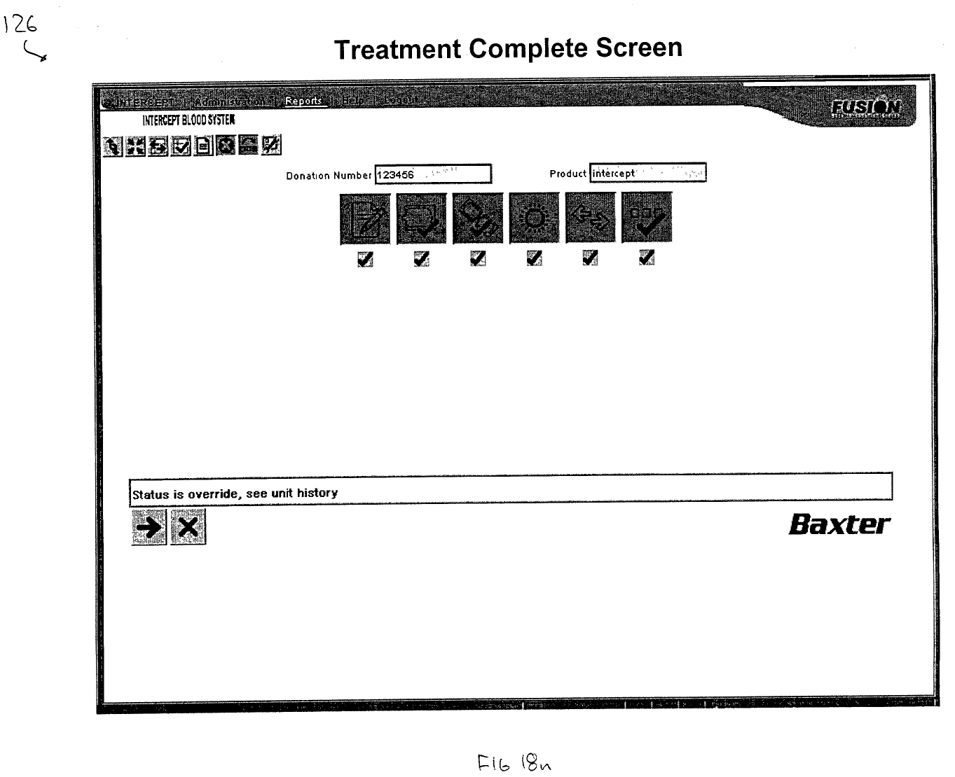

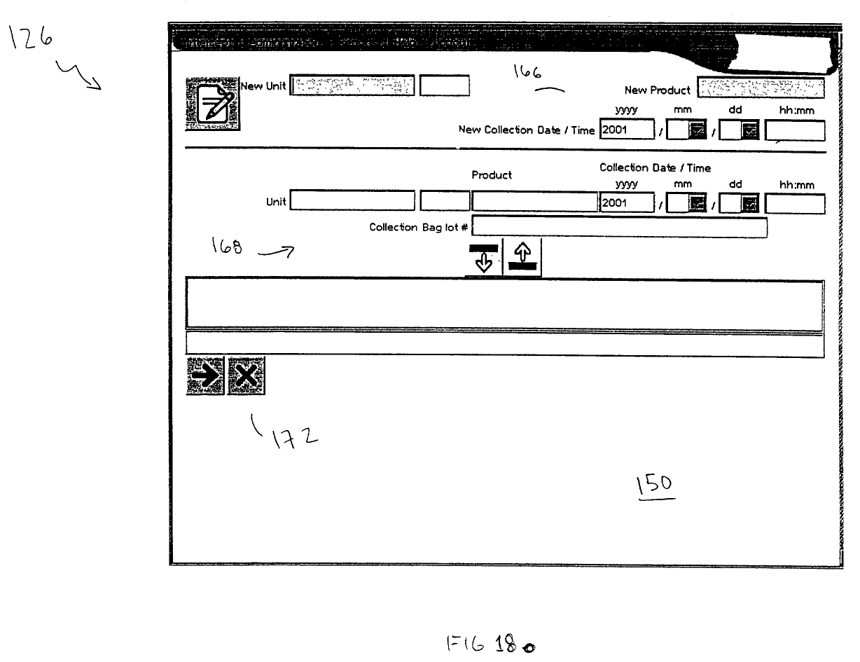

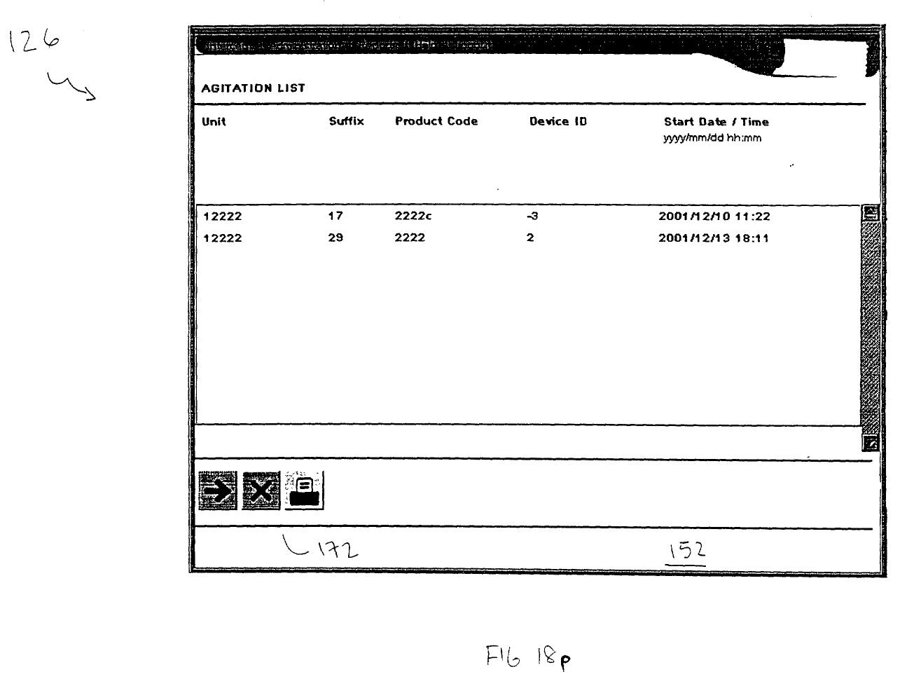

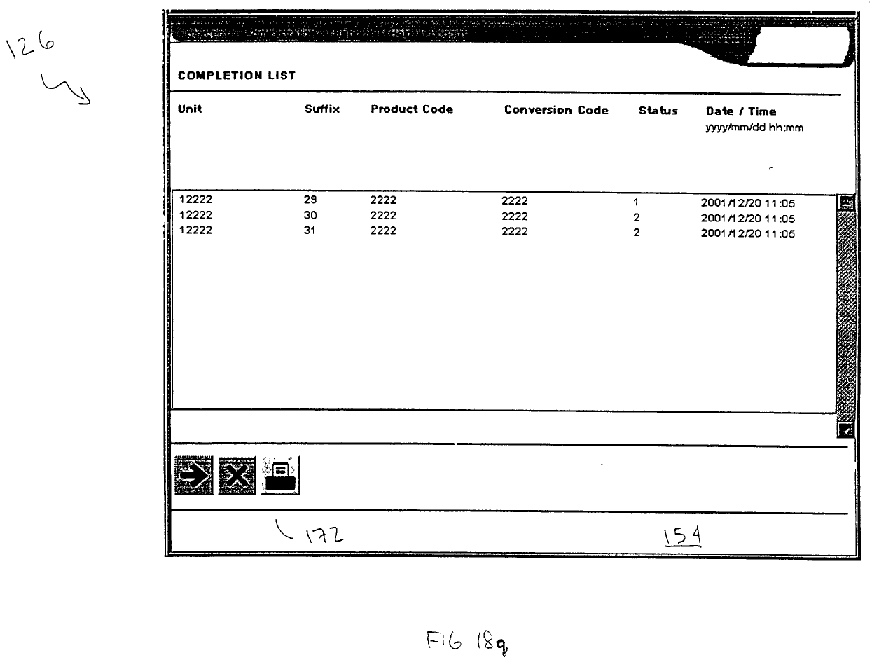

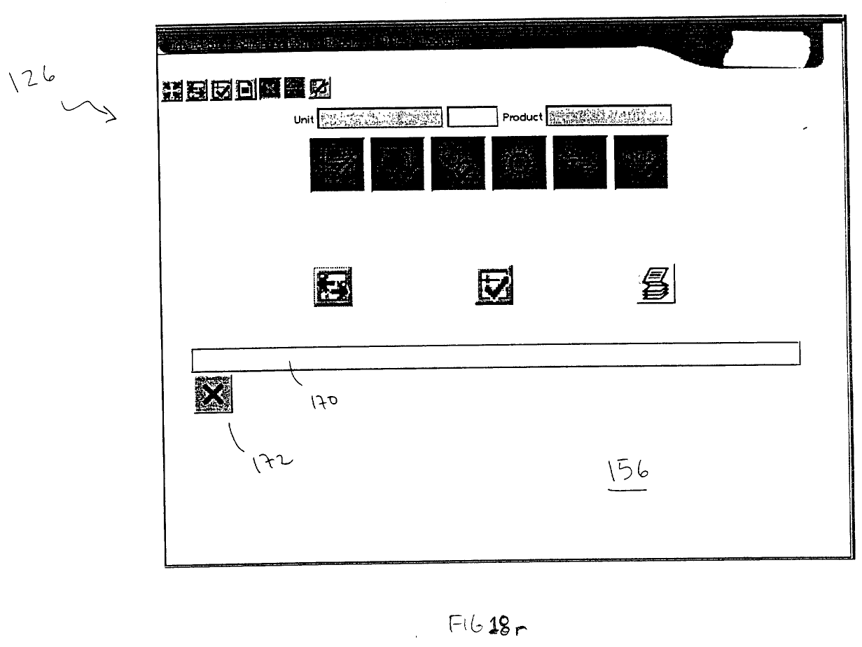

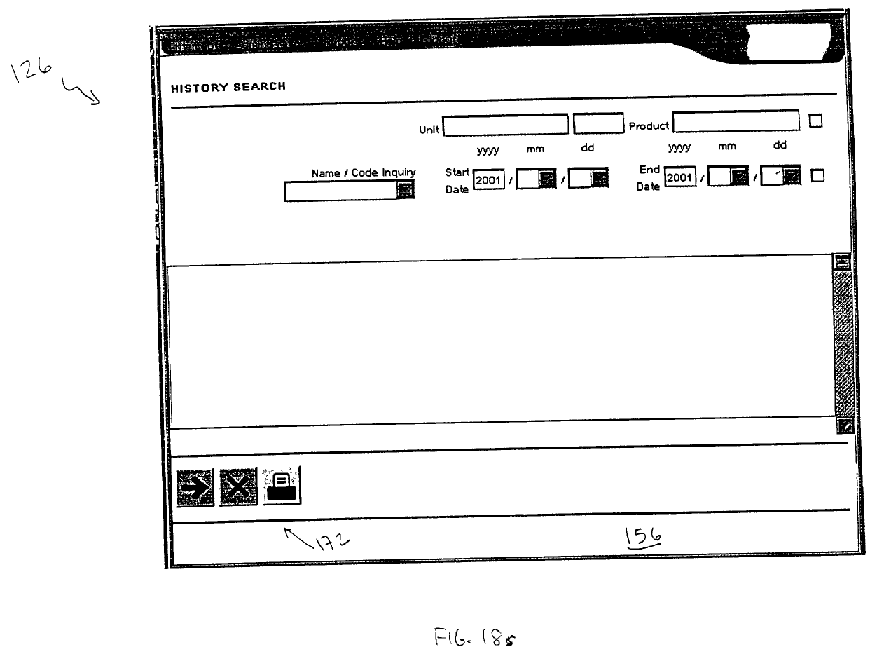

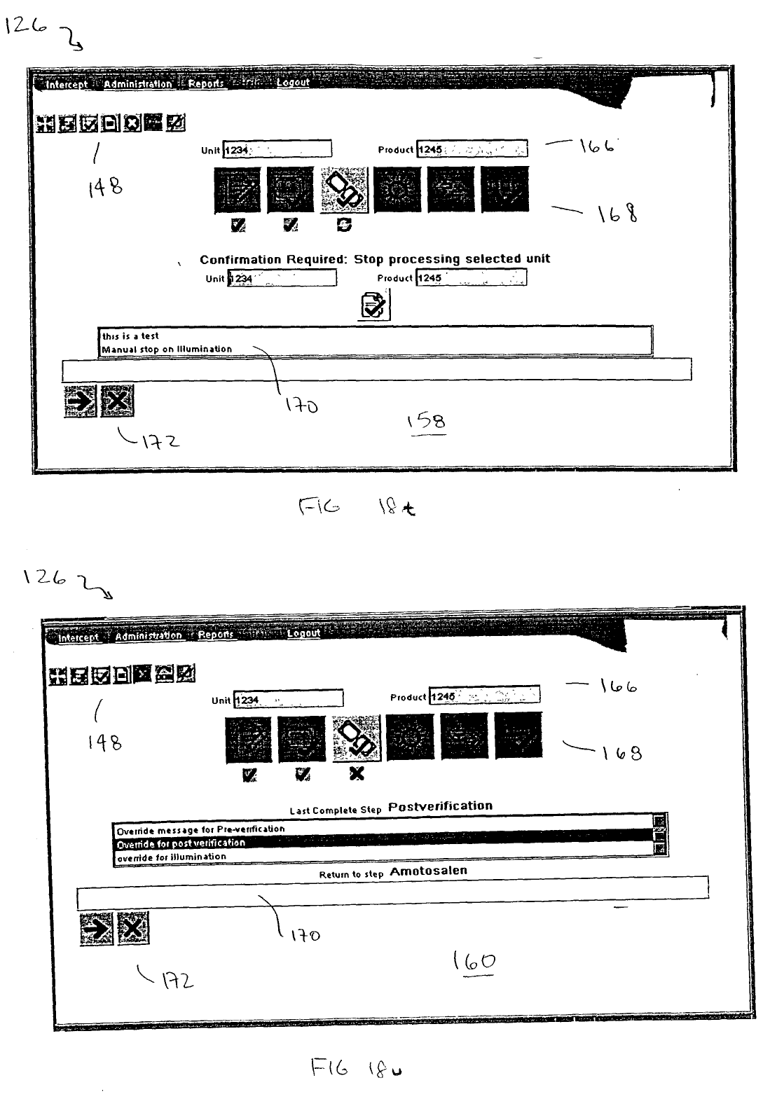

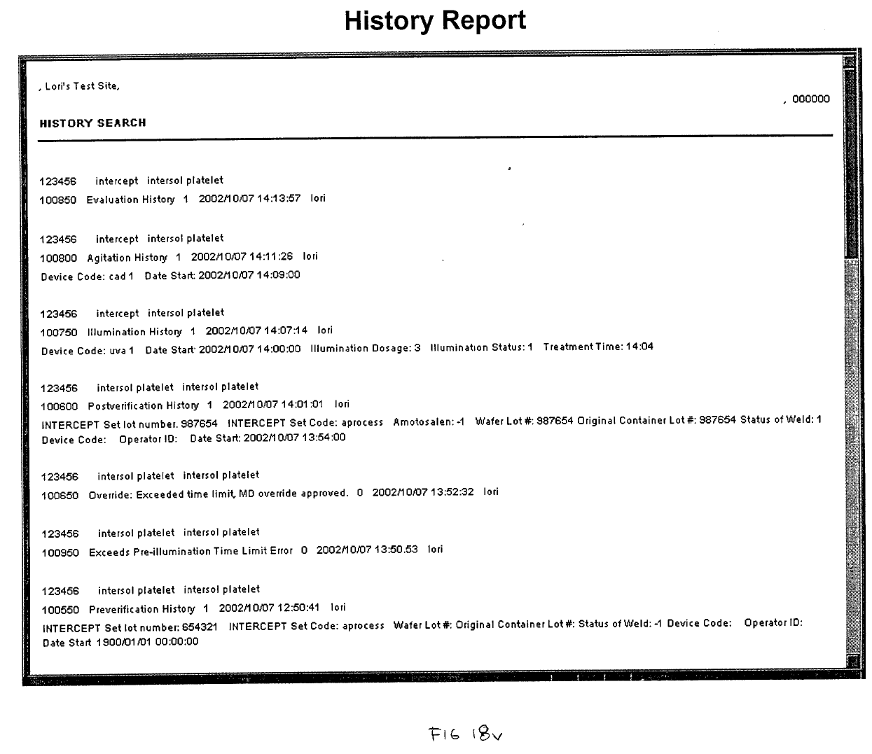

Referring to FIGS. 18a-18v, the user interface 126 enables users to interact with the graphical user displays to input and retrieve data as necessary. The application allows the user to record and retrieve relevant information as well as create reports. The graphical user interface 126 comprises a number of screens designed to achieve specific functions. Each user interface page includes a toolbar 148 (or equivalent) having buttons that perform specific functions. As seen in FIG. 14, the toolbar accesses functions such as: pooling information 150, agitation list 152, completion list 154, reports 156, manual STOP process 158, supervisor override 160, and maintenance tables 162.

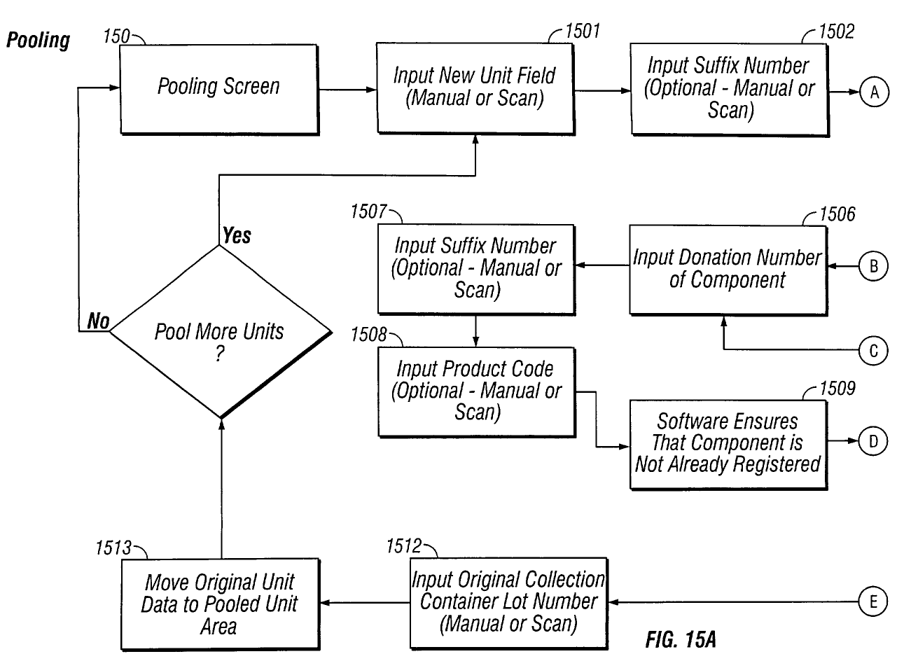

Pooling is generally considered as the tangible and/or intangible combination of physical blood component units (same or different sizes, but of the same type of component) and/or intangible data associated with each of such units. A pooling kit (not shown) can be used to pool blood component units. Pooling information screen 150 enables a user to review previously entered pooling data or creates a new pooling record for multiple smaller units pooled into a single larger unit. FIG. 18o. The pooling data is stored in the system's pool table, but is generally not accessed for any other processes for the pathogen inactivation functions. As shown in FIGS. 15a and b, the pooling screen 150 prompts for the input of a new unit field 1501 (and optionally for a suffix number 1502) and product code of a new unit 1503. The system checks to determine whether the new unit is registered 1504. If the unit is not found, then a new collection date, collection time 1505, donation number 1506, suffix number 1507 (optional), and product code 1508 (optional) can be inputted. The system then checks to see if the component is not already registered 1509. If it has been registered, then an already pooled status 1510 is displayed. If it has not been registered, then the system prompts for the input of the unit collection number 1511 and the container lot number 1512. Using those two identifiers, the system moves the original unit data 1513 into the pooled unit area.

In one embodiment, a pre-processing step is required. This is to determine whether a correct minimal level of blood volume is needed for proper processing of the blood component. In particular, in one embodiment of PI, the amount of platelets in a unit as a percentage of plasma must be in a certain percentage range for proper pathogen inactivation to occur. A preparation kit can be used to establish this proper volume. After verification of the minimal blood volume level, two other preprocessing variables must be properly set: the pre-processing adapter code and the acceptable final product code. In an alternative embodiment of the present invention, product specific variables such as centrifugation and resuspension data fields may also be required. If the pre-processing step requirements are not met, a preprocessing warning message will display. A unit that has successfully completed preprocessing record, results in an “okay” symbol appearing in the Pre and Post Verification indicators for the user screen. Registration can now proceed.

An agitation list 152 enables the user to move to a screen display that lists the units currently in the agitation stage highlighting those that have not met or exceeded predetermined parameters to have completed the minimum amount of hours of exposure to the adsorption wafers. FIG. 18p.

The completion list 154 displays a list of units that have been processed in the last 24 hours and currently have a status other than incomplete. FIG. 18q. Clicking on the report button can generate reports 156. FIGS. 18r and 18s. Predetermined reporting capabilities may be made available, such as pooling data reports, PI completion lists, and process override logs.

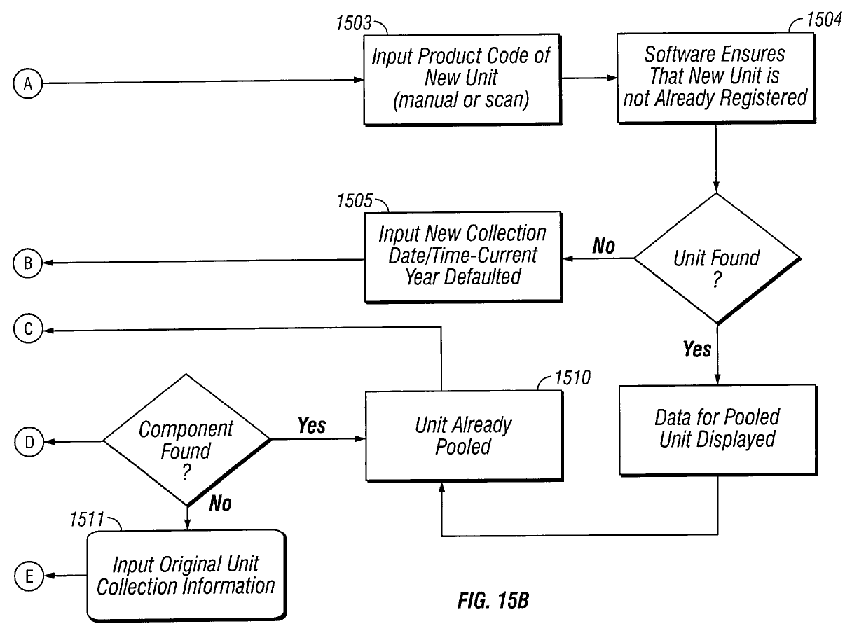

As shown in FIG. 16, a manual STOP process 1600 allows the user to force a manual stop at any point during the treatment. In FIG. 18t, one embodiment of the manual stop screen 158 is shown. To initiate a manual STOP, a unit is located 1602 using a donation number, optional suffix number, and any number of other product fields 1601. Once found, a user confirms 1603 the manual stop by reentering the donation number and product code 1604 for that particular treatment. If there is a match, the user is required to select a reason 1606 for the manual STOP. After completion of the manual STOP process, a unit is placed on manual STOP status and marked incomplete 1607.

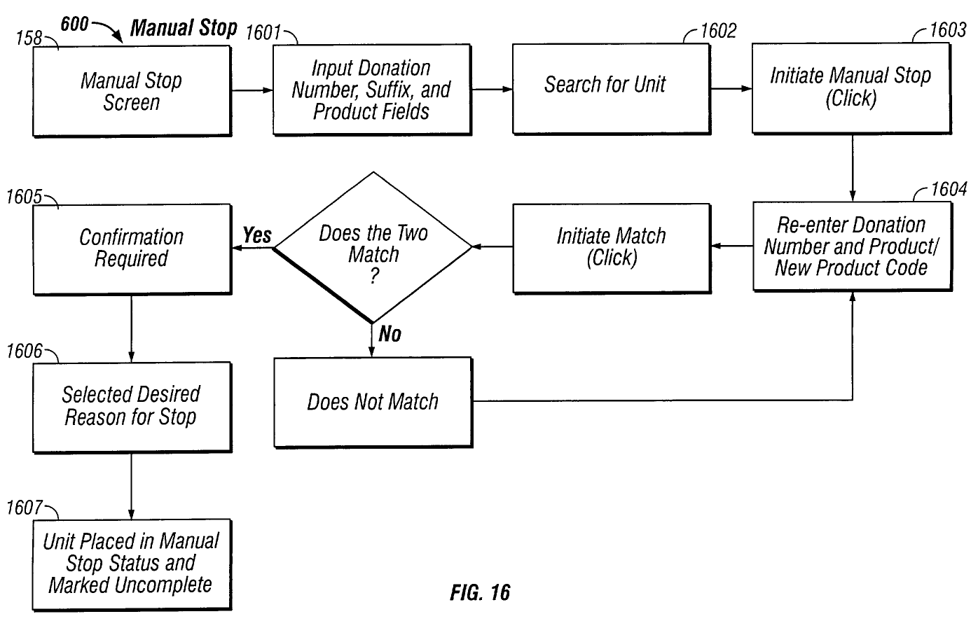

As shown in FIG. 17, the supervisor override process 1700 enables a process halted using a forced STOP 1701 or MANUAL STOP 1702 be restarted at the point when the forced STOP or MANUAL STOP was initiated 1703. FIG. 17. Before an override can be completed, the user access level 1704 is checked to determine if an override is allowed. As shown in FIG. 18u, one embodiment of an override screen 160 requires the user to select the reason for the supervisor to override.

A main database 23 is stored in memory 130 and has a data schema 200 for storing the data in a relational format. One example of a relational data schema 200 is shown in FIG. 19. At the time a new user is created, permissions are defined for that user by assigning a default group code from a base data table, PermGroups. The default configuration for the group code is loaded from the base data table, PermsCFG. Specific permissions for users can be adjusted from the Permissions base data table. Permissions can be viewed through the language base data table, UserPerms.

FIG. 19 depicts one embodiment of the database structure of the present invention. The following list provides a brief description of the various tables utilized within the system database in the memory 130:

classsteps—defines the steps that are required for each class type

devicedata—holds the data retrieved from a device (electronically or manually)

devices—holds the list of devices used by site

history—holds all history event/transactions and the current characteristics at the time of event/transaction

historycodes—holds the descriptions of history events translated into each user=s language

hostdata—holds communication between software and the Host system

messages—holds the display messages translated into each user=s language

permgroups—holds the list of available user groups

permissions—holds the descriptions of permissions translated into each user=s language

permscfg—holds the default permission setting by user group

pimsclasses—holds the list of product classes and their descriptions

pools—holds the data that tracks unit pooling history

products—holds the characteristics and requirements recorded and evaluated in the PI process by product

productXpikit—dictates which PI kits are appropriate for each product and used in evaluation of proper matches

prompts—holds the prompts translated into each user=s language

sites—holds all the site-specific configuration settings

status—holds the current status data from each step of each unit

units—holds all the current characteristics of each unit

userhistory—holds the user history prompts described in the user=s language

usermessages—holds all the user messages described in the user=s language

userperms—holds the user permissions described in the user=s language

userprompts—holds the user prompts as described by the user=s language

users—holds both inactive and active users of the software

The user interface 126 interacts with the database schema 200 to enter and retrieve data as necessary. The user is allowed to record and retrieve relevant information and create reports. The user interface includes several screens designed to facilitate several specific functions. Navigation throughout the user interface is similar to standard accepted conventional practice. For instance, the tab key is used to accept the data in a field and to move the cursor to the next data field. The mouse 31 can be used to move the cursor to different data fields. Pressing the space bar or clicking the mouse will create (or remove) a check mark in a check box. A down arrow on the right side of the data field will indicate pull-down list boxes. Buttons are activated by a mouse click or a A<tab>+<Enter>@ if assigned to the tab sequence.

Initially, the user may be required to log into the system using a login page 173. As shown in FIG. 18a, the login page 173 can include the attributes of a user login prompt 1731, a password prompt 1732, and one or more navigation control icons 1733.

After logging into the system, the user is brought to a general main page 1800. As shown in FIG. 18b, this screen can include basic attributes of a standard pathogen inactivation toolbar 148, a unit identification area 166, step buttons 168, message area 170, and control buttons 172.

The general process for treating the biological fluid, e.g., pathogen inactivation, requires that the biological fluid unit be identified so the current or next step can be determined. Preferably, the unit identification includes a unit number, e.g., donation identity, and a product code. This information is typically obtained from the product container of the treated biological fluid via a bar-code reader 33. When the product code has been entered into the identification field 166, a search of the main database 23 is initiated to determine whether a current record exists. If so, the current status is displayed as further described below. If there is no match, a registration screen appears, FIG. 18f. The step buttons 168 are inactive unless a unit has been identified. The registration screen comprises the basic attributes, in addition to a lot number and treatment kit code.

In one embodiment, a pre-processing screen 1800 as shown in FIG. 18d can be used to determine the correct minimal level of blood volume needed for processing the blood component. This is an optional step occurring prior to the registration process. Attributes of this screen can include: initial volume 1901, centrifuge 1902, conditioning solution variable 1903, resuspension 1904, final volume 1905, preprocessing adapter 1906, lot number 1907, final product code 1908. FIG. 18e shows a completed and checked preprocessing screen 1910 where the indication to proceed is activated in the form of a brightened icon 1911.

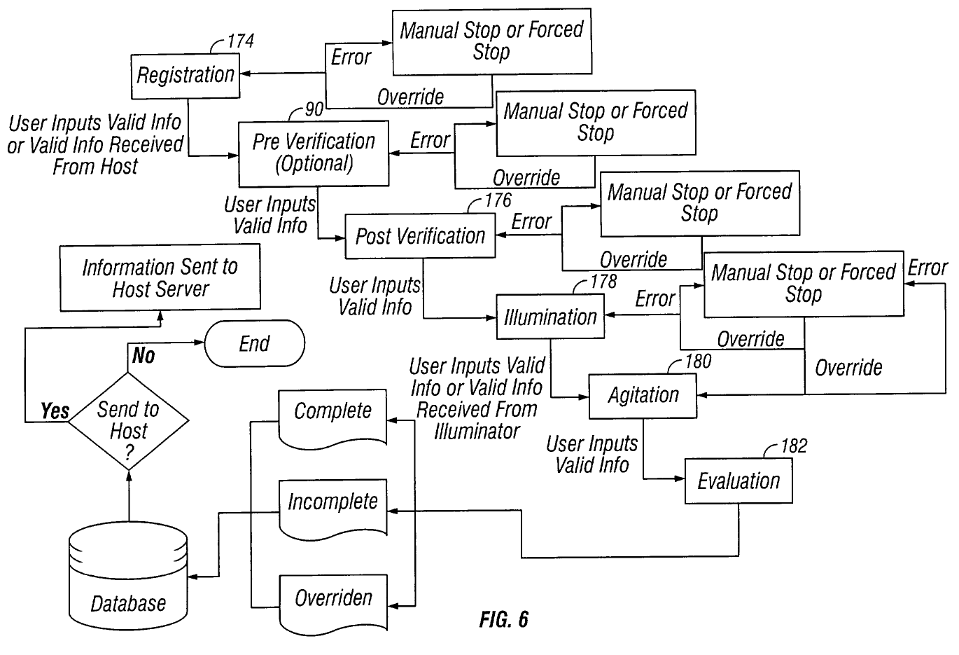

As shown in FIG. 6, the biological treatment process generally includes a plurality of process steps. For the purpose of explanation, one specific biological treatment process C pathogen inactivation C will be discussed. The pathogen inactivation process can include one or more of the following process steps: pooling of a unit; registration of a new unit 80; pre-verification 90; sterile docking; post-verification 100 of a match between the product and the pathogen inactivation kit; illumination 110; agitation 120; and evaluation 1300. Graphical buttons displayed at the user interface 126 represents these process steps. Actuation of a specific button will cause the lower part of the screen to reflect the data and/or information necessary to complete that selected process step.

Each utilized process step is associated with a status condition reflecting the state of the treatment, i.e., complete, in progress, stopped. The lack of a status depicts either that no specific unit has been identified or that a process step is yet to be performed and has no status. An attempt to perform a process step out of sequence may result in a notification, e.g., alarm. Future process steps are not accessible for data entry until the step becomes the current in progress step. As the unit progresses through the pathogen inactivation treatment, a status graphic will update with information related to that particular process step. Review of treatment information associated with a completed step can be acquired by selecting the appropriate process step graphic. Once a process step has been completed, editing its status is not allowed. Only inquiries can be made.

Below the process step icon button is a framed step variable area. The type of information displayed within the frame will change to reflect the step status as well as the site-defined parameters. A message area is used to provide communication notifying operator personnel of input errors, process errors, or overall communication.

Also included on each display page of the interface 126 is a list of control buttons 172. The control buttons 172 facilitate data communication throughout the system by saving current data, evaluating data, or assisting the user to navigate through the system. Additional control buttons 172 can be used for printing reports, and/or moving selected data into and out of list boxes.

Referring to FIG. 18c, the main screen 1810 of the interface 126 is displayed after the user has successfully logged into the system. The main screen 1810 includes a toolbar 148 having basic attributes including: unit identification area 166, process step buttons 168, message area 170, and control buttons 172. From this page, the user has the ability to navigate the interface 126 via the control buttons 172, review the general process steps, or search for a specific product unit.

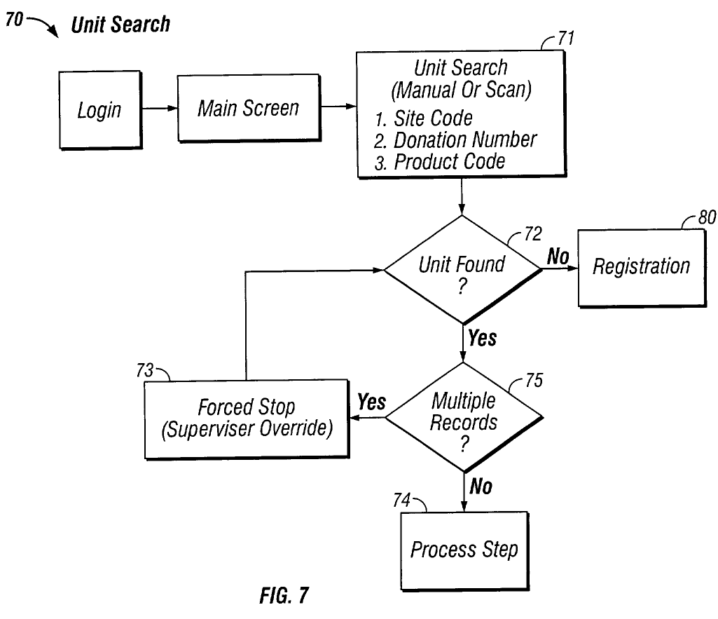

Referring to FIG. 7, the investigation of a specific unit can be accomplished by the unit search process 70. Within a site, a unit is searched on donation number, and product code 71. Keyboard entry 32 or bar-code reading 33 can make the input. The code 71 is checked 72 against the product code table in a database stored in the memory 130. If multiple matching records are returned 75, the process is stopped 73 and requires supervisor attention. If the proper record is matched, the record is evaluated for the current status of each treatment step. The status of each step is then displayed along with the highlighted button of the current step and its corresponding definition 74. A successful match can be used to trigger a search of the Hostdata table 1911 for related information. The Host can send information 2011 such as collection date, time, platelet count, and volume. This information is stored in the hostdata table 1911.

If a biological fluid unit was not matched during the search, the registration process 80 is initiated. The registration page 174, or screen, as shown in FIG. 18f, will be displayed. The page includes the pathogen inactivation toolbar 148, unit identification area 166, processes step buttons 168, message area 170, and control buttons 172. A step variable area 162 contains data fields for the collection date, time, volume and platelet count. Also included on the registration page 174 are check boxes for rest period, active period, and no contamination of red cells as required in the product type and site configuration settings. The collection date and time are evaluated against the current date and time 81 to determine if any time-related warning messages should be displayed or transmitted. For instance some biological fluid products, products data table, have a warning time (uvwarntime), a time limit (uvtimelimit), or a time configuration (uvtimelimitCFG). The UV time limit is the oldest age a product can be prior to UV exposure. If a unit's age exceeds the UV time limit 83, the process treatment of the unit is stopped 82.

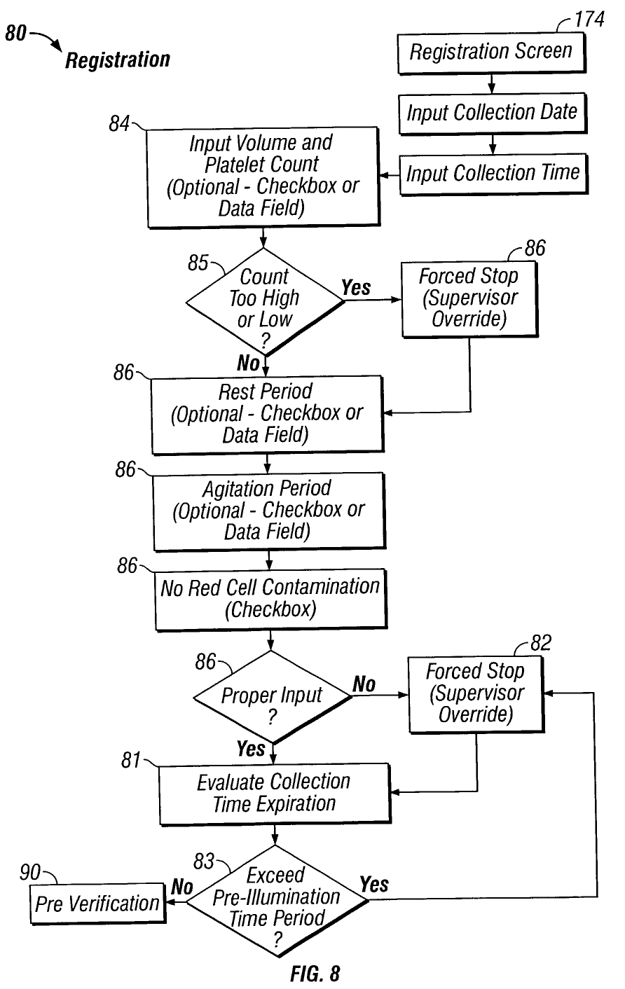

Referring to FIG. 8, various other data fields are configurable during the registration process 80. The product's volume amount 84 can be checked and recorded for quality assurance purposes. Additionally, the product's platelet level 84 can be checked and recorded. These values can be stored and compared 85 with predetermined values in the main database 130, producttable. If during the evaluation, the values are outside the limits of the predetermined values, the process treatment is stopped 86. The check boxes for monitoring rest period, active period, and red cell contamination 86 can be utilized to maintain records and perform quality assurance checks of the biological fluid treatment process. The user has the ability to utilize any combination of these process checking techniques to ensure the quality of the treatment process. At the end of a successful registration process, the registration process will be designated as complete.

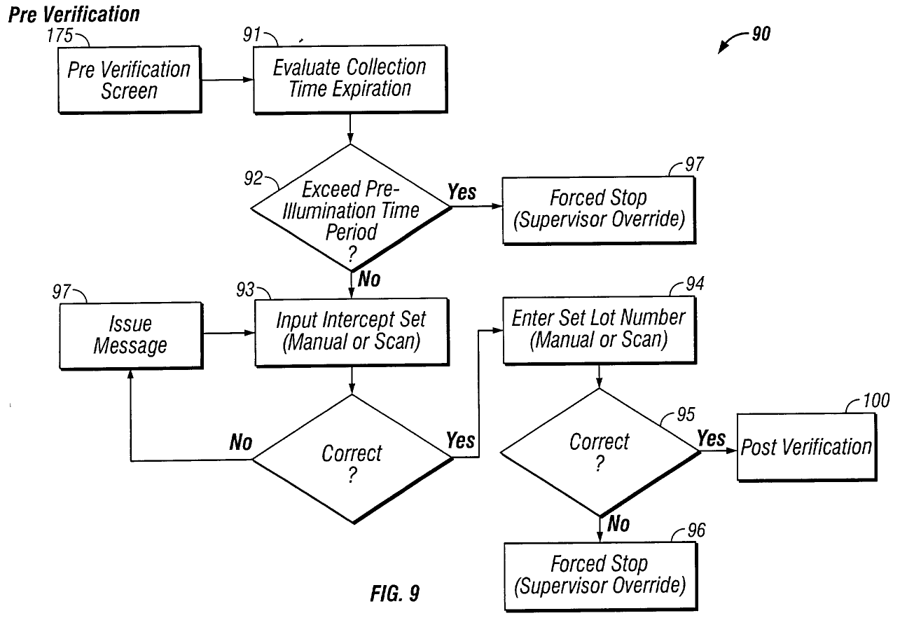

The next step of the pathogen inactivation treatment may be the pre-verification process 90 as shown in FIG. 9. Pre-verification allows operator personnel to confirm the union of a particular product unit with an intended pathogen inactivation kit. Execution of the pre-verification process 90 generates a history transaction and updates the unit data table with the last successful entry pair. The optional pre-verification process can only be executed if the registration process 80 has been completed. The display screen 175 accompanying this step includes the PI toolbar 148, unit identification area 166, process step buttons 168, message area 170, and control buttons 172.

The pre-verification process 90 evaluates the collection time and expiration 91 to determine if the limit has been exceeded 92. If the time limit has been exceeded, the treatment process is stopped 97. Next, the user enters the product lot number 94 and treatment kit number 93 for storing into the productXpikit data table. This is to determine whether the product lot and treatment kit are compatible 95. If either of these inputs is incorrect, an issue message 97 will request correction. The results of this determination are displayed to the user. If the product lot number and treatment kit number are incompatible, a forced stop 96 occurs. The end of a successful execution of a pre-verification process 90 results in a designation of complete.

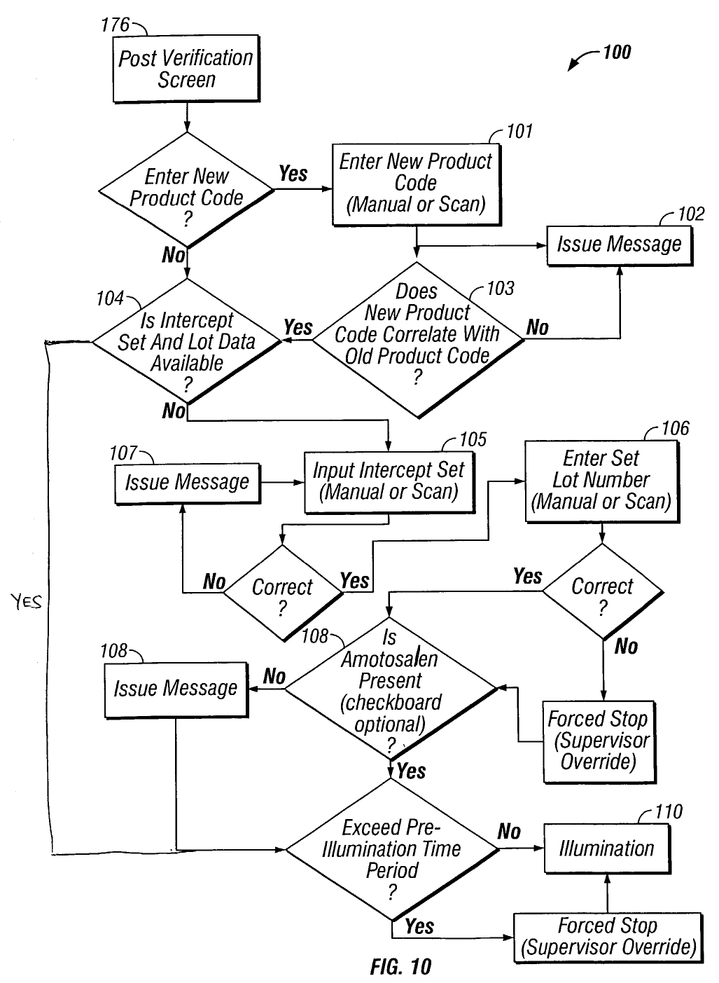

Similar to the optional pre-verification process 90, the mandatory post-verification process 101 shown in FIG. 10 is accessible after a successful registration process 80 or pre-verification process 90. In FIG. 18h, the post-verification screen 176 displays the PI kit code and lot number that is docked to the biological fluid unit. If this information is not available or cannot be found, the PI kit code 105 and lot number 106 can be inputted using a keyboard or barcode scanner. If the entry is incorrect, an issue message 107 will prompt for correction. This process is preferably initiated after physical docking occurs as an assurance of a proper match between the kit code and the lot number. Since the original container with the old product code may have been discarded during the treatment process, a new product code 101 can be inputted. If the new product code does not match 103 the old product code, then an issue message 102 prompts for collection. If desired, a prompt for the light-activated photochemical or photoactive agent, e.g., amotosalen, can be made. The user indicates the presence of amotosalen 108 by clicking the appropriate check box. Although the user can choose whether to evaluate compatibility of the product code and kit, and/or the presence of amotosalen, the preferred requirement for a successfully completed post-verification process step requires a compatible match of the product code and the PI kit code, and the recording of the presence of amotosalen. FIG. 18i shows an example of a post-verification screen 176 with completed data fields.

As part of the post verification process, an alternative embodiment of the present invention requires sterile dock post-verification step if the presence of a sterile dock instrument is detected. One such sterile dock instrument is described in U.S. patent application Publication Ser. No. 10/008,361. The site will determine via maintenance which additional data fields should be presented in this step. The data fields may be pre-populated from information in the device data table received from the sterile dock instrument using FTP protocol or manually entered. Manual entry allows the user to continue with the pathogen inactivation process when the sterile dock instrument is non-functioning. Typically performed after physical docking has occurred, this step ensures that a proper match between the pathogen inactivation kit and the lot number has been made.

The next process in the pathogen inactivation treatment is the illumination process 178. An illumination screen 178 is shown in FIG. 18j. This screen will be displayed when a unit has completed the UVA exposure and a record of the biological fluid unit has been automatically transmitted to and stored in the database in the memory 130. Similar to many of the process display pages discussed above, the illumination display page includes a PI toolbar 148, unit identification area 166, process step buttons 168, message area 170, process step variable area 162, and control buttons 172. The process step variable area 162 contains data fields populated with information obtained from the devicedata data table. This information was recorded during the illumination process 178 for each of the items received from the illumination device C the data fields 111 include: treatment date, device, start time, treatment time, dosage, and status. If the time 117 prior to illumination exceeds the predetermined amount, a forced stop 112 occurs. The treatment date, device, start time, and treatment time can be entered into the main database via bar-coding or numeric entry. The dosage data field reflects the amount of joules exposure measured internally by the illumination instrument 10 and is a true indicator that the unit was properly exposed. The illumination instrument 10 has requirements defining the determination of UVA exposure. These requirements are based on the size and type of product being processed. The data records associated with the illumination can be retrieved 114 and reviewed 113. If multiple records 116 are found, a forced stop 115 occurs resulting in an incomplete status or an invalid match designation. Upon successfully executing the illumination process 110, the status of the illumination process is designated as complete. FIG. 18k shows an example of an illumination screen 178 with completed data fields.

The agitation process 120 may follow successful completion of the illumination process 110 described above. FIG. 18l depicts a display page 180 for the agitation process. The interface includes the PI toolbar 148, unit identification area 166, process step buttons 168, message area 170, process step variable area 162, and control buttons 172. The process variable area 162 contains data fields for documenting the agitation process 120 and includes device identification, start date and start time. A report tool is capable of identifying biological fluid units qualified to continue to the next process. The settings for the agitation process are product-specific and various fields can be presented at the same site. A list of these options includes, agitation time-frame, partial agitation evaluation, and full agitation evaluation. Any combination of these options can be utilized by the user. The data obtained for populating the fields of these options can be manually entered by the user or received by the agitation device. Upon successful execution of the agitation step 120, the status of the agitation step is designated as complete. An agitation list shown in FIG. 18p provides quick access to any unit that has exceeded the minimum time requirement in the agitation/CAD step. If a full evaluation is not required 122 during the agitation process, a prompt for inputting the start time, start date, and other data fields 123 may be made available. The information is then verified 124 and confirmed 125. The contents of the list are updated each time the agitation function list is selected and units are evaluated in response to this selection.

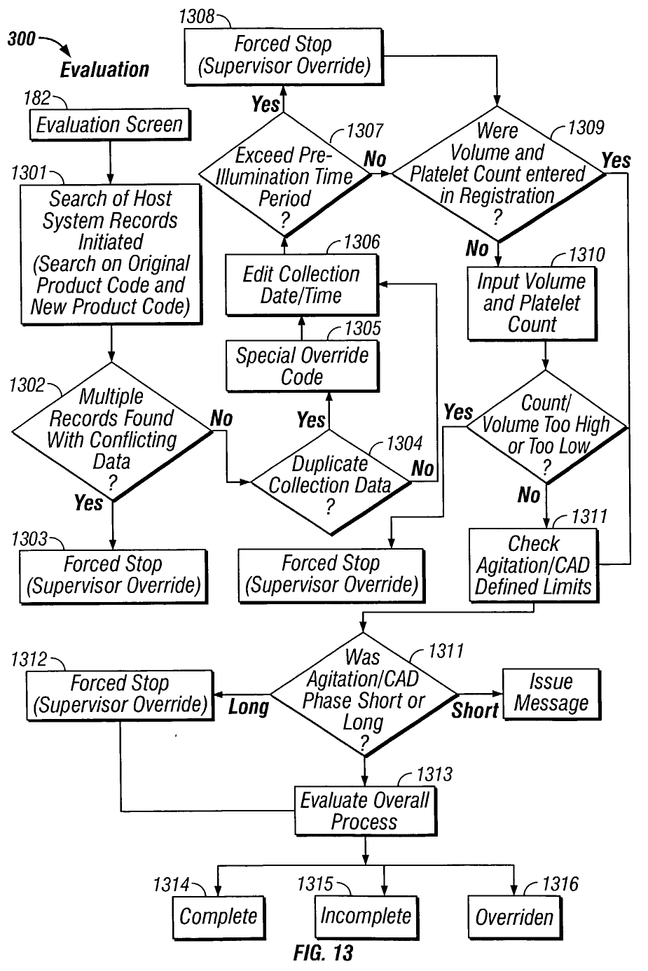

Referring to FIG. 13, when all the required process steps for the pathogen inactivation treatment have been successfully completed, the evaluation process 1300 begins. The evaluation screen 182 as shown in FIG. 18m includes the PI toolbar 148, unit identification area 166, process step buttons 168, message area 170, process step variable area 162, and control buttons 172. The process step variable area 162 contains data fields for documenting date/time and any data missing that are required for final process evaluation of the biological fluid unit. The evaluation process begins by searching the host system records 1301 replicated on the local database using the original product code or a new product code (if issued during the post-verification process). If multiple records 1302 or duplicate records 1304 are found, a forced stop 1303 occurs. Using a special override code 1305, the forced stop can be removed. The user has the ability to manage transfer time/date of the unit's storage, along with the unit's volume and platelet count. During the evaluation process, the data from previous processes are checked. The pre-illumination time 1307 is checked to determine whether the limit has been exceeded. If the limit has been exceeded, a forced stop 1308 occurs. The volume and platelet count from the registration process is checked 1309. If there is no data, a prompt for the input of the data 1310 is provided. The agitation/CAD process is checked to determine if the duration was longer or shorter than predefined limits 1311. If the agitation process was too short, then the collection date and collection time can be modified 1306. If the agitation process was too long, then a forced stop 1312 occurs. Such mechanisms and information facilitate quality assurance related to the pathogen inactivation treatment 1313. At the end of the evaluation process, the treatment is marked with a complete 1314, incomplete 1315, or overridden status 1316.

Similar to the agitation list, a completion list shown in FIG. 18q provides quick access to a list of those units that have successfully completed the pathogen inactivation treatment. The contents of the list are updated each time the completion function list is selected and each biological fluid unit is evaluated in response to this selection. The pages shown in FIGS. 18r and 18s can be utilized for generating reports from the contents at each step in the pathogen inactivation treatment.

Maintenance of the system is generally focused on three areas: site, product, and device. Various maintenance tables can be accessed through the toolbar 148. Site maintenance primarily involves access and overall parameters. Product maintenance enables the site to enter specific definitions for each product code as well as establish the appropriate reference to pathogen inactivation kits. Device maintenance also concerns the types of devices connected to the system.

Site maintenance includes three interrelated forms: general parameters, permission groups, and user forms. General parameters involve data utilized for reports and interfaces, and also relate to determinations regarding the function of prompts in separate process step sections. The implementation of various permission groups enables the site to assign different levels of access to system personnel and streamline new user information entry.

Product maintenance involves two forms: user-defined product codes and several specific product parameters. The product codes are assigned to a product class and associated with acceptable PI kits. The defined product codes and parameters are referenced to determine whether the correct product and treatment kit are docked together. The second form relates to the grouping classifications and the site requirements for the class. The user form is completed for each unique profile that is desired to obtain access permissions and tracking levels.

Permission group maintenance provides a mechanism to efficiently add users using a standard hierarchy of permission groups. Preferably, two groups are predefined and additional groups can be created. The codes created allow operator personnel to enter several permission levels with reduced key strokes. The group and permission code fields have a lookup feature via a pull-down menu. The group codes can be utilized to edit an existing record. The permission code/description field is a lookup of the permission code with an accompanying description.

The general parameters are divided into several sections: site identification, site communication, Host communication, and function definitions. A general parameter form must be completed before a report is able to correctly identify the related site.

The product maintenance form must be completed before a biological fluid unit can enter the PI treatment so that the barcode will be recognized and the unit will be processed accurately. Sections on the form relate to product grouping, site-definable parameters, and product code—PI kit relationship. A device definition form is utilized to establish device codes for the system's devices, e.g., UVA Illuminator, agitators, etc. Several product classes are predefined. A pull-down menu form is utilized to configure the system's process steps. A list displays the available process steps for the configuration. The selected process steps are displayed according to the order of expected performance.

In one embodiment, a device interface facilitates communication between a system device and the system. As shown in FIGS. 2, 4, and 5, the interface utilizes standard FTP protocol parameters wherein the system acts as the FTP server and the device acts as the FTP client. Other protocols can be used. Information related to the device is sent with each data transmission.

A Host interface facilitates communication between the Host and the system. FTP records sent by the Host to the system are stored in the hostdata data table. Other protocols can be used as well. The table is reviewed whenever a record search of the blood component unit is performed. The data is used to populate the fields as described above. The system will search the hostdata data table during the evaluation step 182 so that data entered in the Host at a later point is transferred to the unit data table.

As mentioned above, the present invention further provides the ability to pool units. At least two units must be combined to constitute a pool. A unique number is assigned to the pooled product and is utilized for tracking. A pooling screen page shown in FIG. 18o is displayable by the UI 126 and includes the PI toolbar, new unit identification area, registration step button, and control buttons. The original unit identification, collection information and collection container, e.g., bag, lot number are entered for each product that is added to the pool. The new unit identification is entered as described earlier in the registration screen process. Both manual and bar-coded entries are acceptable. The product codes, before and after pooling, should be defined within the products data table. The oldest date of the blood component units to be pooled is selected as the new unit collection date. The collection bag lot number can be entered manually or via bar-coding. The list of units in the pool can be viewed by scrolling vertically through a display box on the pooling screen. After a pool has been created and documented, the user can directly access the registration screen by clicking the step button. The new unit identification area will populate the appropriate registration fields for unit identification and collection information.

The present invention also provides a manual stop process wherein the user is allowed to stop the process for a reason other than a treatment step failure. These reasons may include: container leak, temperature failure, damaged/mishandled blood component unit, etc. Notification of a manual stop can be displayed in the user message area and the step status area of the display page shown in FIG. 18t. Preferably, not all users are permitted to manually stop the treatment. This will be based on the permissions of the group accessible by the user.

A blood component unit may fail a pathogen inactivation treatment step, but still remain viable for continuation of the treatment provided a proper assessment has been conducted. Such ability is preferably restricted to supervisory and/or medical personnel. A very high level of security should be associated with such a capability wherein designated personnel are authorized to perform this function. An override feature is suitable in certain circumstances, such as the docking of the wrong PI kit. Because a stop status will cause the blood component unit to be discarded, the override allows a correctable situation to be rectified and the unit to be regained. FIG. 18u.

To summarize, the present invention is suited for medical facilities where product integrity and traceability are critical quality factors. The instruments, laboratory equipment and data input devices can be connected to an Ethernet network along with other data processing applications. A computer acting as a server/gateway executes applications to receive the transmitted data and route them to the database in the memory 130 and hypertext markup language (HTML) applications.

Operator personnel can perform data queries and reporting on a local area network, a wide area network, over the Internet, or any combination thereof, using a standard browser application interface. Real-time viewing and updating of device operation can be configured for any number of devices on the browser. In addition, the server also transmits encrypted data to a wireless personal digital assistant (PDA). The PDA's OS executes a standard application browser interface for viewing portable information, alarms, and other event notifications. The Pads are also used for data input (through a keypad touch screen, scanning, or other entering method C all used interchangeably herein) in association with an apparatus operation. Thus, the present invention includes open standard architecture in a heterogeneous apparatus environment with real-time updating and accessing of data, and portable data viewing, reporting, notification, and inputting.

Depending on the steps utilized during the biological fluid treatment, a plurality of graphical buttons is utilized to represent each process step. Addressing the preferred embodiment of the present invention, the steps of the pathogen inactivation treatment are represented by a plurality of buttons, i.e., registration, post-verification, illumination, agitation, and evaluation. Actuation of each button will display a related page reflecting data and information associated with the process step. If a blood component unit has been identified, the data and information provided will reflect the specific status information for the identified unit.

One embodiment of the system of the present invention is designed for the treatment of a blood component. The general purpose of the system is to provide a process control or process audit mechanism to ensure proper execution of treating biological fluids, e.g., pathogen inactivation of blood and blood components. This purpose is fulfilled principally through coordinating management of information during the treatment process and interaction with system instruments, e.g., illuminator. Previously, operator personnel were required to manually keep track of such information, but the present invention allows personnel to omit such manual involvement. The system may also provide some of the following benefits: improved accuracy and completeness in the data for each step in the pathogen inactivation process for a particular biological fluid product; increased data collected for diagnostic use, which may give rise to better information with which to design or troubleshoot system instruments; increased data collected for use by the facility for generation of ad-hoc statistical reports, which could relate any number of variables such as units which have completed the pathogen inactivation process; units which have not completed the process and history of completed steps and incomplete steps; greater efficiency throughout the treatment process due to less paperwork; decreased costs due to less office paperwork; ability to research all the detailed information of a treatment, or the history of an individual blood component unit, accurate monitoring of the facility procedures; collection of information that may assist personnel to improve system efficiency and overall workflow.

It will be understood that the invention may be embodied in other specific forms without departing from the spirit or central characteristics thereof. The present embodiments, therefore, are to be considered in all respects as illustrative and not restrictive, and the invention is not to be limited to the details given herein.

What is claimed is:

1. A system for blood product treatment, the system comprising:
a processor;
a memory being operably connected to the processor, the memory storing a blood product treatment module;
at least one blood product treatment instrument for communicating information related to blood product treatment to the processor and memory over a communications network for treating a blood product; and,
an interface having means for entering information, means for displaying information, and means for communicating with the processor and memory, the information being related to the blood product treatment, wherein the processor, the memory, and the interface are for at least one of monitoring, managing, reporting, and administering the blood product treatment.

2. The system of claim 1, wherein the interface comprises at least one of a personal digital assistant, a desktop computer, a laptop computer, and a wireless handheld computer.

3. The system of claim 1, wherein the means for displaying the information comprises a graphical screen.

4. The system of claim 1, wherein the means for entering the information comprises at least one of a keyboard, a bar-code reader, and a mouse.

5. The system of claim 1, wherein the blood product treatment comprises at least one of a platelet treatment, a plasma treatment, a red blood cell treatment, and a double red blood cell treatment.

6. The system of claim 1, wherein the blood product treatment comprises pathogen inactivation.

7. The system of claim 6, wherein the pathogen inactivation utilizes a photoactive agent.

8. The system of claim 7, wherein the treatment instrument is an illuminator.

9. The system of claim 8, wherein the blood product treatment module operates with the illuminator using a pathogen inactivation kit and the photoactive agent to inactivate pathogens in the blood product.

10. The system of claim 1, wherein the blood product treatment module receives pathogen inactivation kit data and receives blood product type data, and determines whether the pathogen inactivation kit data is compatible with the blood product type data.

11. The system of claim 10, wherein a compatibility message is sent to the interface indicating whether the pathogen inactivation kit data is compatible with blood product type data.

12. The system of claim 1, wherein the blood product treatment module registers blood product registration data for at least one unit of the blood product.

13. The system of claim 12, wherein the blood product registration data is received by at least one of an interface and a host, and is sent to a database in the memory.

14. The system of claim 13, wherein the blood product treatment module records blood product treatment data for storage in a database in the memory.

15. The system of claim 14, wherein the blood product treatment data is generated by an illuminator.

16. The system of claim 13, wherein the blood product treatment module evaluates blood product treatment data to ensure proper execution of a blood product treatment.

17. The system of claim 16, wherein the blood product treatment module generates a status report of an evaluation of a blood product treatment.

18. The system of claim 13, wherein the blood product treatment module determines that a predetermined time limit for processing of a blood product has met at least one of is approaching, has been reached, and has been exceeded.

19. The system of claim 18, wherein the predetermined time limit is one of at least a time for collection, a time for pre-illumination procedures, a time for illumination, a time for agitation, and a time for post-illumination procedures.

20. The system of claim 18, wherein the blood product treatment module generates a stop message for the blood product treatment indicating that the predetermined time limit has been reached or exceeded.

21. The system of claim 18, wherein the blood product treatment module generates a time limit message indicating that the predetermined time limit is one of is approaching, has been reached, and has been exceeded.

22. The system of claim 18, wherein the blood product treatment module stops the blood product treatment after the blood product treatment module has determined that the predetermined time limit has been reached or exceeded.

23. The system of claim 22, wherein the blood product treatment module receives a command to override a stopped blood product treatment.

24. The system of claim 1, wherein the blood product treatment module receives a blood product identifier.

25. The system of claim 1, wherein the blood product treatment module identifies the blood product.

26. The system of claim 1, wherein the blood product treatment module stops a blood product treatment process.

27. The system of claim 26, wherein the blood product treatment module overrides stopped blood product treatment.

28. The system of claim 1, wherein the blood product treatment module locates the treatment instrument using at least one data field.

29. The system of claim 28, wherein the data field comprises at least one of a site code, a donation number, a suffix, and a product code.

30. A method for at least one of monitoring, reporting, managing, and administering a blood product non-apheresis treatment, the method comprising the steps of:
providing for registering a blood product; and,
providing for storing blood product treatment information from a treatment instrument.

31. The method of claim 30, further comprising the step of:
providing for verifying the compatibility of the blood product with a blood product treatment kit.

32. The method of claim 30, further comprising the step of:
providing for stopping a blood product treatment process.

33. The method of claim 32, further comprising the step of:
providing for overriding the stopped blood product treatment process.

34. The method of claim 30, further comprising the step of:
providing for evaluating the blood product treatment to ensure proper administration of treatment steps.

35. The method of claim 30, further comprising the step of:
providing for storing sterile connect device information.

36. For a blood product treatment system, the system comprising a memory having a database and a blood product treatment module, a processor operably connected to at least one treatment instrument and to the memory, and an interface operably connected to the processor for facilitating a blood product treatment, a method for at least one of monitoring, reporting, managing, and administering a blood product treatment comprising the steps of:
providing for receiving a blood product identifier identifying a blood product; and,
providing for receiving a treatment kit identifier identifying a treatment kit.

37. The method of claim 36 further comprising the step of:
providing for determining whether the blood product and the treatment kit are compatible for use.

38. The method of claim 37, wherein the blood product treatment module compares the blood product identifier with the treatment kit identifier.

39. The method of claim 36, further comprising the step of:
providing for stopping the administration of the blood product treatment.

40. The method of claim 39 wherein stopping the administration of the blood product treatment is in response to the blood product treatment module determining that the treatment kit and the blood product are incompatible.

41. The method of claim 36, further comprising the step of:
providing for overriding a stopped blood product treatment.

42. The method of claim 36, further comprising the step of:
providing for storing information about a stopped blood product treatment process.

43. The method of claim 36, further comprising the step of:
providing for storing information about a stopped blood product process override.

44. The method of claim 36, further comprising the step of:
providing for storing collection information about the blood product, the collection information comprising a blood product type and a time of collection.

45. The method of claim 36, further comprising the steps of:
providing for calculating a time limit, the time limit comprising a date and a time of a blood product collection plus a fixed amount of time.

46. The method of claim 45, further comprising the step of:
providing for stopping administration of the blood product treatment in response to the blood product treatment module determining that the time limit has been reached or exceeded.

47. The method of claim 36, further comprising the step of:
providing for stopping administration of the blood product treatment in response to the blood product treatment module determining that a cell count for the blood product is less than a predetermined cell count level.

48. The method of claim 36, further comprising the step of:
providing for selecting an illuminator device to treat the blood product.

49. The method of claim 36, further comprising the step of:
providing for determining whether all necessary steps prior to illumination have been completed.

50. The method of claim 36, further comprising the step of:
providing for sending an illumination message to the interface indicating that an illumination step is ready to be completed, for inactivating pathogens within the blood product.

51. The method of claim 36, further comprising the steps of:
providing for receiving information from the treatment instrument, the information comprising an illumination exposure count transmitted by the treatment instrument.

52. The method of claim 51, further comprising the step of:
providing for determining if the illumination exposure count has reached or exceeded an illumination exposure limit.

53. The method of claim 52, further comprising the step of:
providing for stopping execution of the blood product treatment in response to the illumination exposure exceeding the illumination exposure limit.

54. The method of claim 36, further comprising the step of:
providing for generating an illumination limit message indicating that predetermined illumination exposure limit is one of is approaching, has been reached, and has been exceeded.

55. The method of claim 36, further comprising the step of:
providing for agitation of the blood product.

56. The method of claim 36, further comprising the steps of:
providing for determining whether a sorption time is one of at least approaching, reaching, or exceeding a predetermined sorption time.

57. The method of claim 56, wherein sorption is at least one of adsorption and absorption.

58. The method of claim 56, wherein the predetermined sorption time limit is a minimal duration for agitating the blood component.

59. The method of claim 36, further comprising the step of:
providing for generating a sorption limit message indicating that a predetermined sorption limit is one of is approaching, has been reached, and has been exceeded.

60. The method of claim 56, further comprising the step of:
providing for stopping execution of the blood product treatment in response to the determination that the sorption time has reached or exceeded the predetermined sorption time.

61. The method of claim 36, further comprising the step of:
providing for evaluating the blood product treatment.

62. The method of claim 61, further comprising the step of:
providing for reporting the evaluation of a completed blood product treatment.

63. The method of claim 36, further comprising the step of:
providing for monitoring the administration of blood product treatment.

64. The method of claim 36, wherein the step of providing for registering the blood product further comprises the steps of:
providing for prompting an entry of a blood product identifier;
providing for prompting an entry for a treatment kit identifier;
providing for receiving an input to the prompt for the blood product identifier, and,
providing for receiving an input to the prompt for the treatment kit identifier.

65. The method of claim 36, further comprising the step of:
providing for locating the treatment instrument using at least one data field.

66. The method of claim 65, wherein the data field comprises at least one of a site code, a donation number, a suffix, and a product code.

67. The method of claim 36, wherein the blood product treatment further comprises the step of:
registering the blood product using at least one identifier.

68. The method of claim 67, wherein the at least one identifier comprises at least one of a collection date and a collection time.

69. The method of claim 68, wherein the at least one identifier further comprises at least one of a volume amount, a platelet count, a rest period, an agitation period, and a red cell contamination status.

70. The system of claim 36, further comprising the step of:
providing for receiving blood product information from a host source.

71. The method of claim 70 wherein the blood product information is at least one of a platelet count and a volume collection amount.

72. The method of claim 36, further comprising the steps of:
providing for initiating a stop mechanism for stopping the blood product treatment in response to a platelet count being at least one of higher and lower in relation to a predetermined platelet count; and,
providing for initiating an override mechanism for overriding the stopped blood product treatment.

73. The method of claim 36, further comprising the steps of:
providing for initiating a stop mechanism for stopping the blood product treatment in response to a volume collection amount being at least one of higher and lower in relation to a predetermined volume collection amount; and,
providing for initiating an override mechanism for overriding the stopped blood product treatment.

74. The method of claim 36, further comprising the step of:
providing for determining whether the blood product identifier is invalid.

75. The method of claim 36, further comprising the step of:
providing for determining whether the treatment kit identifier is invalid.

76. The method of claim 36, further comprising the step of:
providing for converting the blood product identifier to a new blood product identifier using a conversion code.

77. The method of claim 76, further comprising the step of:
providing for sending a message indicating that an incorrect conversion code was used; and,
providing for initiating a correction mechanism to allow for correcting the conversion code.

78. The method of claim 36, further comprising the step of:
providing for sending data regarding the blood product treatment of a blood product to a host system.

79. A computer readable medium having an application therein for at least one of monitoring, reporting, managing, and administering a blood product treatment of a blood product, the medium comprising:
a first segment for receiving blood product identification information; and,
a second segment for tracking the status of the blood product treatment of the blood product throughout the blood product treatment by reference to blood product treatment identification information.

80. The medium of claim 79, further comprising:
a third segment for verifying compatibility of the blood product with a pathogen inactivation kit.

81. The medium of claim 79, further comprising:
a third segment for receiving information from a pathogen inactivation instrument utilized during treatment, the information relating to an amount of exposure of illumination transmitted from the pathogen inactivation instrument to the blood product; and,
a fourth segment for storing the information.

82. The medium of claim 81, further comprising:
a fifth segment for determining whether the amount of illumination exposure satisfies a predetermined amount of illumination.

83. The medium of claim 82, further comprising:
a sixth segment for stopping the treatment of the blood product if the measured amount of illumination is less than the predetermined level of illumination.

84. The medium of claim 79, further comprising:
a third segment for overriding a stopped blood product treatment.

85. The medium of claim 84, further comprising:
a fourth segment for determining a time duration between a collection of the blood product and illumination of the blood product.

86. The medium of claim 85, further comprising:
an fifth segment for stopping the treatment of the blood product in response to the time duration reaching or exceeding a predetermined collection time amount.

87. The medium of claim 79, further comprising:
a third segment for evaluating a completed blood product treatment wherein a status of the completed blood product treatment is determined.

88. The medium of claim 79, further comprising:
a third segment for receiving information in a product form; and,
a fourth segment for storing information received from the product form into a database stored in a memory.

89. The medium of claim 79, further comprising:
a third segment for generating a report, the report comprising information related to the blood product.

90. The medium of claim 79, further comprising:
a third segment for determining whether a platelet count of the blood product is acceptable.

91. The medium of claim 79, further comprising:
a third segment for determining whether a blood product volume is acceptable.

92. The medium of claim 79, further comprising:
a third segment for registering the blood product.

93. The medium of claim 79, further comprising:
a third segment for sending a message notifying that a failure to satisfy a predetermined treatment requirement has taken place.

94. The medium of claim 79, further comprising:
a third segment for maintaining integrity of a blood product wherein a conversion code is utilized in cooperation with a blood product identifier for the blood product.

95. The system of claim 1 wherein data received from the treatment instrument regarding aspects of the blood product treatment is maintained in a database.

96. The system of claim 1 wherein current status data of each step in the blood product treatment is maintained in a database.
